(19) United States
(12) Patent Application Publication
(10) Pub. No.: US 2009/0118180 A1
(43) Pub. Date: May 7, 2009

(54) USE OF FUSION PROTEINS FOR THE PREVENTION OR THE TREATMENT OF PATHOLOGIES RESULTING FROM ISCHEMIA

(75) Inventors: Brigitte Onteniente, Creteil (FR); Christelle Guegan, Paris (FR); Jerome Braudeau, Paris (FR); Cecile Couriaud, Paris (FR)

Correspondence Address: YOUNG & THOMPSON, 209 Madison Street, Suite 500, ALEXANDRIA, VA, 22314, US

(73) Assignee: Institut National De La Sante ET De La Recherche Medicale (Inserm), Paris (FR)

(21) Appl. No.: 12/088,725

(22) PCT Filed: Sep. 29, 2006
(86) PCT No.: PCT/EP2006/009495

§ 371 Date: Jun. 11, 2008

(30) Foreign Application Priority Data
Sep. 29, 2005 (EP) 05292028.7

(51) Int. Cl. A61K 38/17; A61K 38/02; C07K 14/47; A61K 38/08

(52) U.S. Cl. 514/12; 514/2; 514/15; 530/350

(57) ABSTRACT

The present invention relates to the use of a fusion protein including a peptide sequence having membrane transducing properties, and a peptide sequence having cell survival properties, or a nucleic acid coding for the fusion protein, for the manufacture of a medicament intended for the prevention or the treatment of pathologies resulting from ischemia.

The present invention relates to the use of a fusion protein, or a nucleic acid coding for said fusion protein, for the manufacture of a medicament intended for the prevention or the treatment of pathologies resulting from ischemia.

Cumulative evidence that caspases actively contribute to ischemia-induced brain lesions (Friedlander, 2003) has fostered important developments in pre-clinical research (Onteniente, 2004). However, the limitation of neurological failure by synthetic caspase inhibitors administered after the onset of ischemia has mainly been obtained in the case of mild ischemic insults (Plesnila and Moskowitz, 2000), and further developments are in progress to improve their stability, specificity and efficacy. In addition, considering the complexity of ischemic cell death mechanisms, efficient cell protection is unlikely to be provided by inhibition of caspases only.

Major endogenous inhibitors of caspases are the members of the IAP family. XIAP is its most potent member (Vaux and Silke, 2003), and has a greater caspase inhibitory capacity than synthetic peptides (Deveraux et al., 1999), very likely related to its complex structure. XIAP possesses three baculovirus IAP repeat (BIR) domains, two of which (BIR2 and BIR3) interact with caspases, and a COOH terminal really interesting new gene (RING) zinc-finger motif endowed with E3 enzyme activity (Yang et al., 2000) that marks targets for degradation in the ubiquitin/proteasome pathway, among which are caspase-3 and XIAP itself (Huang et al., 2000). Besides caspase inhibition, XIAP is also involved in the modulation of several signaling pathways (Lewis et al, 2004), and activates DNA binding of the transcription factor NF-κB (Holcik et al., 2001). This multimodal action gives XIAP a unique neuroprotective efficacy, and overexpression of XIAP appears a promising strategy to counteract lesions with complex etiology, as stroke and ischemic cardiopathies.

XIAP is a 62 kDa protein that does not cross the blood brain barrier (BBB). As a result, the neuroprotective potential of XIAP in stroke has been analyzed with virally mediated overexpression (Xu et al., 1999), or using transgenic mice (Trapp et al., 2003).

The main goal of the present invention is to provide pharmaceutical compositions conferring membrane transducing properties to peptide sequences having cell survival properties, said pharmaceutical compositions being intended for the prevention or the treatment of pathologies resulting from ischemia, and more particularly resulting from myocardial or cerebral ischemia.

The invention relates to the use of:
a fusion protein comprising or constituted of:
   at least one peptide sequence having membrane transducing properties, and
   at least one peptide sequence having cell survival properties; or
a homologous protein derived from said fusion protein by insertion, deletion, or substitution of at least one amino acid, provided that said homologous protein presents an identity percentage of at least 85%, and preferably of at least 89%, with respect to said fusion protein and presents membrane transducing and cell survival properties, or
a nucleic acid sequence coding for said fusion protein or for said homologous protein,
   for the manufacture of a medicament intended for the prevention or the treatment of pathologies resulting from ischemia.

Transducing properties mentioned above can be assessed according to Dowdy et al. (Methods 24:247-256, 2001), by labeling the fusion protein directly with fluorescein isothiocyanate (FITC) or with immunohistochemistry using anti-HA antibodies followed by fluorescent secondary antibodies, and counting the cells that have incorporated the fusion protein with fluorescence-activated cell sorting.

Cell survival properties mentioned above can be assessed in vitro by adding the protein to cell cultures after induction of cell death by apoptotic agents, hypoxia or oxygen-glucose deprivation, and quantification of the number of apoptotic cells by means of apoptosis specific markers such as externalization of phosphatidylserine or production of lactate dehydrogenase, or by quantifying the number of surviving cells. Cell survival properties can be assessed in vivo by quantification of the number of surviving cells in a tissue after induction of degeneration by a variety of stimulus that include ischemia, apoptosis inducers or traumatic insults, or by quantification of the global volume of tissue loss.

Cell survival properties include caspase inhibiting properties. Said caspase inhibiting properties can be assessed in vitro by adding the protein to substrates specific for caspases in vitro, or by reaction of extracts from tissues treated with the protein to specific caspase substrates (Benchoua et al., J. Neurosci. 21:7127-7134, 2001).

The invention relates more particularly to the use of a fusion protein as described above, or of a homologous protein derived from said fusion protein as defined above, or of a nucleic acid sequence coding for said fusion protein or for said homologous protein, for the manufacture of a medicament intended for the prevention or the treatment of pathologies resulting from myocardial ischemia.

The invention more particularly concerns the use for the manufacture of a medicament intended for the prevention or the treatment of pathologies resulting from myocardial ischemia, of a fusion protein as defined above wherein the peptide sequence having membrane transducing properties is the protein transduction domain (PTD) of the HIV TAT protein.

The invention relates more particularly to the use for the manufacture of a medicament intended for the prevention or the treatment of pathologies resulting from myocardial ischemia, of a fusion protein as defined above wherein the peptide sequence having membrane transducing properties is the protein transduction domain (PTD) of the HIV TAT protein represented by SEQ ID NO: 2.

The invention concerns more particularly the use for the manufacture of a medicament intended for the prevention or the treatment of pathologies resulting from myocardial ischemia, of a fusion protein as defined above wherein the peptide sequence having cell survival properties is selected from the list comprising:
the X chromosome-linked apoptosis inhibitor protein (XIAP) or fragments thereof having cell survival properties, such as the BIR2, BIR3-RING, or BIR3-RING linker domains of XIAP, or
the Flice inhibitory protein (FLIP) or fragments thereof having cell survival properties.

XIAP or FLIP proteins, or fragments thereof, preferably are mammals proteins, and more preferably human proteins.

The BIR2 domain of the XIAP protein is the second BIR domain of XIAP associated to the linker sequence between BIR1 and BIR2. The BIR2 domain possesses caspase inhibitory properties.

The BIR3-RING domain of the XIAP protein is the association of the third BIR domain of XIAP and the Really Interesting New Gene zinc finger domain, including the linker region between both domains.

The BIR3-RING linker domain is the region that separates the BIR3 and RING domains of XIAP.

The invention concerns more particularly the use for the manufacture of a medicament intended for the prevention or the treatment of pathologies resulting from myocardial ischemia, of a fusion protein as defined above wherein the peptide sequence having cell survival properties is selected from the list comprising:
the XIAP represented by SEQ ID NO: 4 (rat),
the XIAP represented by SEQ ID NO: 6 (human),
the BIR2 domain represented by SEQ ID NO: 8 (rat),
the BIR2 domain represented by SEQ ID NO: 44 (human),
the BIR3-RING domain represented by SEQ ID NO: 10 (rat),
the BIR3-RING domain represented by SEQ ID NO: 12 (human),
the BIR3-RING linker domain represented by SEQ ID NO: 36 (rat),
the BIR3-RING linker domain represented by SEQ ID NO: 38 (human),
the FLIP represented by SEQ ID NO: 14 (FlipL) (mouse),
the FLIP represented by SEQ ID NO: 16 (FlipS) (mouse),
the FLIP represented by SEQ ID NO: 18 (Flip control) (mouse).

The invention relates more particularly to the use for the manufacture of a medicament intended for the prevention or the treatment of pathologies resulting from myocardial ischemia, of a fusion protein as defined above wherein the fusion protein is selected from the list comprising:
PTD-XIAP represented by SEQ ID NO: 20, corresponding to the fusion of SEQ ID NO: 2 and SEQ ID NO: 4,
PTD-XIAP represented by SEQ ID NO: 22, corresponding to the fusion of SEQ ID NO: 2 and SEQ ID NO: 6,
PTD-BIR2 represented by SEQ ID NO: 24, corresponding to the fusion of SEQ ID NO: 2 and SEQ ID NO: 8,
PTD-BIR2 represented by SEQ ID NO: 46, corresponding to the fusion of SEQ ID NO: 2 and SEQ ID NO: 44,
PTD-BIR3-RING represented by SEQ ID NO: 26, corresponding to the fusion of SEQ ID NO: 2 and SEQ ID NO: 10,
PTD-BIR3-RING represented by SEQ ID NO: 28, corresponding to the fusion of SEQ ID NO: 2 and SEQ ID NO: 12,
PTD-(BIR3-RING linker) represented by SEQ ID NO: 40, corresponding to the fusion of SEQ ID NO: 2 and SEQ ID NO: 36,
PTD-(BIR3-RING linker) represented by SEQ ID NO: 42, corresponding to the fusion of SEQ ID NO: 2 and SEQ ID NO: 38,
PTD-FLIP represented by SEQ ID NO: 30, corresponding to the fusion of SEQ ID NO: 2 and SEQ ID NO: 14,
PTD-FLIP represented by SEQ ID NO: 32, corresponding to the fusion of SEQ ID NO: 2 and SEQ ID NO: 16,
PTD-FLIP represented by SEQ ID NO: 34, corresponding to the fusion of SEQ ID NO: 2 and SEQ ID NO: 18.

The invention concerns more particularly the use as defined above, of:
a fusion protein comprising or constituted of:
   at least one peptide sequence having membrane transducing properties, and
   at least one peptide sequence having cell survival properties selected from the list comprising:
      fragments of the X chromosome-linked apoptosis inhibitor protein (XIAP) having cell survival properties, or
      fragments of the Flice inhibitory protein (FLIP) having cell survival properties,
a homologous protein derived from said fusion protein by insertion, deletion, or substitution of at least one amino acid, provided that said homologous protein presents an identity percentage of at least 85%, and preferably of at least 89%, with respect to said fusion protein and presents membrane transducing and cell survival properties, or
a nucleic acid coding for said fusion protein or for said homologous protein,
   provided that said fusion protein or said homologous protein does not comprise a whole XIAP sequence,
   for the manufacture of a medicament intended for the prevention or the treatment of pathologies resulting from myocardial ischemia.

The invention preferably relates to the use for the manufacture of a medicament intended for the prevention or the treatment of pathologies resulting from myocardial ischemia, of a fusion protein as defined above wherein the peptide sequence having cell survival properties comprises at least one XIAP fragment having cell survival properties selected from the list comprising:
the BIR2 domain, or
the BIR3-RING domain, or
the BIR3-RING linker domain.

The invention relates more preferably to the use for the manufacture of a medicament intended for the prevention or the treatment of pathologies resulting from myocardial ischemia, of a fusion protein as defined above, wherein the peptide sequence having cell survival properties comprises at least one XIAP fragment having cell survival properties which is the BIR2 domain with the linker domain between the BIR1 and BIR2 domains.

In this respect, the invention concerns more particularly the use for the manufacture of a medicament intended for the prevention or the treatment of pathologies resulting from myocardial ischemia, of a fusion protein as defined above wherein the peptide sequence having cell survival properties is represented by SEQ ID NO: 8 or represented by SEQ ID NO: 44.

The invention relates more particularly to the use for the manufacture of a medicament intended for the prevention or the treatment of pathologies resulting from myocardial ischemia, of a fusion protein as defined above wherein the fusion protein is PTD-BIR2 represented by SEQ ID NO: 24 or PTD-BIR2 represented by SEQ ID NO:46.

The invention relates more preferably to the use for the manufacture of a medicament intended for the prevention or the treatment of pathologies resulting from myocardial ischemia, of a fusion protein as defined above wherein the peptide sequence having cell survival properties comprises at least one XIAP fragment having cell survival properties which is the BIR3-RING domain or the BIR3-RING linker domain.

In this respect, the invention concerns more particularly the use for the manufacture of a medicament intended for the prevention or the treatment of pathologies resulting from myocardial ischemia, of a fusion protein as defined above wherein the peptide sequence having cell survival properties is selected from the list comprising:
the BIR3-RING domain represented by SEQ ID NO: 10,
the BIR3-RING domain represented by SEQ ID NO: 12,
the BIR3-RING linker domain represented by SEQ ID NO:36,
the BIR3-RING linker domain represented by SEQ ID NO:38.

The invention relates more particularly to the use for the manufacture of a medicament intended for the prevention or the treatment of pathologies resulting from myocardial ischemia, of a fusion protein as defined above wherein the fusion protein is selected from the list comprising:
PTD-BIR3-RING represented by SEQ ID NO: 26, corresponding to the fusion of SEQ ID NO: 2 and SEQ ID NO: 10,
PTD-BIR3-RING represented by SEQ ID NO: 28, corresponding to the fusion of SEQ ID NO: 2 and SEQ ID NO: 12,
PTD-(BIR3-RING linker) represented by SEQ ID NO: 40, corresponding to the fusion of SEQ ID NO: 2 and SEQ ID NO: 36,
PTD-(BIR3-RING linker) represented by SEQ ID NO: 42, corresponding to the fusion of SEQ ID NO: 2 and SEQ ID NO: 38.

The invention concerns more particularly the use as defined above, wherein the pathology is myocardial infarction.

The invention also relates to the use of a fusion protein as described above, or of a homologous protein derived from said fusion protein as defined above, or of a nucleic acid coding for said fusion protein or for said homologous protein, for the manufacture of a medicament intended for the prevention or the treatment of pathologies resulting from cerebral ischemia.

The invention relates more particularly to the use for the manufacture of a medicament intended for the prevention or the treatment of pathologies resulting from cerebral ischemia, of a fusion protein as defined above wherein the peptide sequence having membrane transducing properties is the protein transduction domain (PTD) of the HIV TAT protein.

The invention relates more particularly to the use for the manufacture of a medicament intended for the prevention or the treatment of pathologies resulting from cerebral ischemia, of a fusion protein as defined above wherein the peptide sequence having membrane transducing properties is the protein transduction domain (PTD) of the HIV TAT protein represented by SEQ ID NO: 2.

The invention concerns more particularly the use for the manufacture of a medicament intended for the prevention or the treatment of pathologies resulting from cerebral ischemia, of a fusion protein as defined above wherein the peptide sequence having cell survival properties is selected from the list comprising:
the X chromosome-linked apoptosis inhibitor protein (XIAP) or fragments thereof having cell survival properties, such as the BIR2, BIR3-RING, or BIR3-RING linker domains of XIAP, or
the Flice inhibitory protein (FLIP) or fragments thereof having cell survival properties.

The invention concerns more particularly the use for the manufacture of a medicament intended for the prevention or the treatment of pathologies resulting from cerebral ischemia, of a fusion protein as defined above wherein the peptide sequence having cell survival properties is selected from the list comprising:
the XIAP represented by SEQ ID NO: 4 (rat),
the XIAP represented by SEQ ID NO: 6 (human),
the BIR2 domain represented by SEQ ID NO: 8 (rat),
the BIR2 domain represented by SEQ ID NO: 44 (human),
the BIR3-RING domain represented by SEQ ID NO: 10 (rat),
the BIR3-RING domain represented by SEQ ID NO: 12 (human),
the BIR3-RING linker domain represented by SEQ ID NO: 36 (rat),
the BIR3-RING linker domain represented by SEQ ID NO: 38 (human),
the FLIP represented by SEQ ID NO: 14 (FlipL) (mouse),
the FLIP represented by SEQ ID NO: 16 (FlipS) (mouse),
the FLIP represented by SEQ ID NO: 18 (Flip control) (mouse).

The invention relates more particularly to the use for the manufacture of a medicament intended for the prevention or the treatment of pathologies resulting from cerebral ischemia, of a fusion protein as defined above wherein the fusion protein is selected from the list comprising:
PTD-XIAP represented by SEQ ID NO: 20, corresponding to the fusion of SEQ ID NO: 2 and SEQ ID NO: 4,
PTD-XIAP represented by SEQ ID NO: 22, corresponding to the fusion of SEQ ID NO: 2 and SEQ ID NO: 6,
PTD-BIR2 represented by SEQ ID NO: 24, corresponding to the fusion of SEQ ID NO: 2 and SEQ ID NO: 8,
PTD-BIR2 represented by SEQ ID NO: 46, corresponding to the fusion of SEQ ID NO: 2 and SEQ ID NO: 44,
PTD-BIR3-RING represented by SEQ ID NO: 26, corresponding to the fusion of SEQ ID NO: 2 and SEQ ID NO: 10,
PTD-BIR3-RING represented by SEQ ID NO: 28, corresponding to the fusion of SEQ ID NO: 2 and SEQ ID NO: 12,
PTD-(BIR3-RING linker) represented by SEQ ID NO: 40, corresponding to the fusion of SEQ ID NO: 2 and SEQ ID NO: 36,
PTD-(BIR3-RING linker) represented by SEQ ID NO: 42, corresponding to the fusion of SEQ ID NO: 2 and SEQ ID NO: 38,
PTD-FLIP represented by SEQ ID NO: 30, corresponding to the fusion of SEQ ID NO: 2 and SEQ ID NO: 14,
PTD-FLIP represented by SEQ ID NO: 32, corresponding to the fusion of SEQ ID NO: 2 and SEQ ID NO: 16,
PTD-FLIP represented by SEQ ID NO: 34, corresponding to the fusion of SEQ ID NO: 2 and SEQ ID NO: 18.

The invention concerns more particularly the use as defined above, of:
a fusion protein comprising or constituted of:
   at least one peptide sequence having membrane transducing properties, and
   at least one peptide sequence having cell survival properties selected from the list comprising:
      fragments of the X chromosome-linked apoptosis inhibitor protein (XIAP) having cell survival properties, or
      fragments of the Flice inhibitory protein (FLIP) having cell survival properties,
a homologous protein derived from said fusion protein by insertion, deletion, or substitution of at least one amino acid, provided that said homologous protein presents an identity percentage of at least 85%, and preferably of at least 89%, with respect to said fusion protein and presents membrane transducing and cell survival properties, or
a nucleic acid coding for said fusion protein or for said homologous protein,
   provided that said fusion protein or said homologous protein does not comprise a whole XIAP sequence,
   for the manufacture of a medicament intended for the prevention or the treatment of pathologies resulting from cerebral ischemia.

The invention preferably relates to the use for the manufacture of a medicament intended for the prevention or the treatment of pathologies resulting from cerebral ischemia, of a fusion protein as defined above wherein the peptide sequence having cell survival properties comprises at least one XIAP fragment having cell survival properties selected from the list comprising:
the BIR2 domain, or
the BIR3-RING domain, or
the BIR3-RING linker domain.

The invention relates more preferably to the use for the manufacture of a medicament intended for the prevention or the treatment of pathologies resulting from cerebral ischemia, of a fusion protein as defined above wherein the peptide sequence having cell survival properties comprises at least one XIAP fragment having cell survival which is the BIR2 domain with the linker between the BIR1 and BIR2 domains.

In this respect, the invention concerns more particularly the use for the manufacture of a medicament intended for the prevention or the treatment of pathologies resulting from cerebral ischemia, of a fusion protein as defined above wherein the peptide sequence having cell survival properties is the domain represented by SEQ ID NO: 8 or represented by SEQ ID NO: 44.

The invention relates more particularly to the use for the manufacture of a medicament intended for the prevention or the treatment of pathologies resulting from cerebral ischemia, of a fusion protein as defined above wherein the fusion protein is PTD-BIR2 represented by SEQ ID NO: 24 or PTD-BIR2 represented by SEQ ID NO:46.

The invention relates more preferably to the use for the manufacture of a medicament intended for the prevention or the treatment of pathologies resulting from cerebral ischemia, of a fusion protein as defined above wherein the peptide sequence having cell survival properties comprises at least one XIAP fragment having cell survival properties which is the BIR3-RING domain or the BIR3-RING linker domain.

In this respect, the invention concerns more particularly the use for the manufacture of a medicament intended for the prevention or the treatment of pathologies resulting from cerebral ischemia, of a fusion protein as defined above wherein the peptide sequence having cell survival properties is selected from the list comprising:
the BIR3-RING domain represented by SEQ ID NO: 10,
the BIR3-RING domain represented by SEQ ID NO: 12,
the BIR3-RING linker domain represented by SEQ ID NO:36,
the BIR3-RING linker domain represented by SEQ ID NO:38.

The invention relates more particularly to the use for the manufacture of a medicament intended for the prevention or the treatment of pathologies resulting from cerebral ischemia, of a fusion protein as defined above wherein the fusion protein is selected from the list comprising:
PTD-BIR3-RING represented by SEQ ID NO: 26, corresponding to the fusion of SEQ ID NO: 2 and SEQ ID NO: 10,
PTD-BIR3-RING represented by SEQ ID NO: 28, corresponding to the fusion of SEQ ID NO: 2 and SEQ ID NO: 12,
PTD-(BIR3-RING linker) represented by SEQ ID NO: 40, corresponding to the fusion of SEQ ID NO: 2 and SEQ ID NO: 36,
PTD-(BIR3-RING linker) represented by SEQ ID NO: 42, corresponding to the fusion of SEQ ID NO: 2 and SEQ ID NO: 38.

The invention concerns more particularly the use as defined above, wherein the pathology is cerebral infarction.

The invention also relates to a pharmaceutical composition comprising as active substance:
a fusion protein comprising or constituted of:
   at least one peptide sequence having membrane transducing properties, and
   at least one peptide sequence having cell survival properties; or
a homologous protein derived from said fusion protein by insertion, deletion, or substitution of at least one amino acid, provided that said homologous protein presents an identity percentage of at least 85%, and preferably of at least 89%, with respect to said fusion protein and presents membrane transducing and cell survival properties, in association with a pharmaceutically acceptable carrier.

The invention relates more particularly to a pharmaceutical composition as defined above, suitable for the administration to an individual of a unit dose from about 50 mg to about 500 mg of the fusion protein.

The invention concerns more particularly a pharmaceutical composition as defined above, suitable for the administration to an individual of a dose from about 1 mg/kg to about 10 mg/kg of the fusion protein.

The invention relates more particularly to a pharmaceutical composition as defined above, suitable for an administration by the intravenous route to an individual.

The invention concerns more particularly a pharmaceutical composition as defined above, wherein the peptide sequence having membrane transducing properties is the protein transduction domain (PTD) of the HIV TAT protein.

The invention relates more particularly to a pharmaceutical composition as defined above, wherein the peptide sequence having membrane transducing properties is the protein transduction domain (PTD) of the HIV TAT protein represented by SEQ ID NO: 2.

The invention concerns more particularly a pharmaceutical composition as defined above, wherein the peptide sequence having cell survival properties is selected from the list comprising:
the X chromosome-linked apoptosis inhibitor protein (XIAP) or fragments thereof having cell survival properties, such as the BIR2, or BIR3-RING, or BIR3-RING linker domains of XIAP, or
the Flice inhibitory protein (FLIP) or fragments thereof having cell survival properties.

The invention also concerns more particularly a pharmaceutical composition as defined above, wherein the peptide sequence having cell survival properties is selected from the list comprising:
the XIAP represented by SEQ ID NO: 4 (rat),
the XIAP represented by SEQ ID NO: 6 (human),
the BIR2 domain represented by SEQ ID NO: 8 (rat),
the BIR2 domain represented by SEQ ID NO: 44 (human),
the BIR3-RING domain represented by SEQ ID NO: 10 (rat),
the BIR3-RING domain represented by SEQ ID NO: 12 (human),
the BIR3-RING linker domain represented by SEQ ID NO: 36 (rat),
the BIR3-RING linker domain represented by SEQ ID NO: 38 (human),
the FLIP represented by SEQ ID NO: 14 (FlipL) (mouse),
the FLIP represented by SEQ ID NO: 16 (FlipS) (mouse),
the FLIP represented by SEQ ID NO: 18 (Flip control) (mouse).

The invention relates more particularly to a pharmaceutical composition as defined above, wherein the fusion protein is selected from the list comprising:
PTD-XIAP represented by SEQ ID NO: 20, corresponding to the fusion of SEQ ID NO: 2 and SEQ ID NO: 4,
PTD-XIAP represented by SEQ ID NO: 22, corresponding to the fusion of SEQ ID NO: 2 and SEQ ID NO: 6,
PTD-BIR2 represented by SEQ ID NO: 24, corresponding to the fusion of SEQ ID NO: 2 and SEQ ID NO: 8,
PTD-BIR2 represented by SEQ ID NO: 46, corresponding to the fusion of SEQ ID NO: 2 and SEQ ID NO: 44,
PTD-BIR3-RING represented by SEQ ID NO: 26, corresponding to the fusion of SEQ ID NO: 2 and SEQ ID NO: 10,
PTD-BIR3-RING represented by SEQ ID NO: 28, corresponding to the fusion of SEQ ID NO: 2 and SEQ ID NO: 12,
PTD-(BIR3-RING linker) represented by SEQ ID NO: 40, corresponding to the fusion of SEQ ID NO: 2 and SEQ ID NO: 36,
PTD-(BIR3-RING linker) represented by SEQ ID NO: 42, corresponding to the fusion of SEQ ID NO: 2 and SEQ ID NO: 38,
PTD-FLIP represented by SEQ ID NO: 30, corresponding to the fusion of SEQ ID NO: 2 and SEQ ID NO: 14,
PTD-FLIP represented by SEQ ID NO: 32, corresponding to the fusion of SEQ ID NO: 2 and SEQ ID NO: 16,
PTD-FLIP represented by SEQ ID NO: 34, corresponding to the fusion of SEQ ID NO: 2 and SEQ ID NO: 18.

The invention concerns more particularly a pharmaceutical composition as defined above comprising as active substance:
a fusion protein comprising or constituted of:
   at least one peptide sequence having membrane transducing properties, and
   at least one peptide sequence having cell survival properties selected from the list comprising:
      fragments of the X chromosome-linked apoptosis inhibitor protein (XIAP) having cell survival properties, or
      fragments of the Flice inhibitory protein (FLIP) having cell survival properties,
a homologous protein derived from said fusion protein by insertion, deletion, or substitution of at least one amino acid, provided that said homologous protein presents an identity percentage of at least 85%, and preferably of at least 89%, with respect to said fusion protein and presents membrane transducing and cell survival properties, provided that said fusion protein or said homologous protein does not comprise a whole XIAP sequence, in association with a pharmaceutically acceptable carrier.

The invention also relates more particularly to a pharmaceutical composition as defined above, wherein the peptide sequence having cell survival properties comprises at least one XIAP fragment having cell survival properties selected from the list comprising:
the BIR2 domain, or
the BIR3-RING domain, or
the BIR3-RING linker domain.

The invention also relates more particularly to a pharmaceutical composition as defined above, wherein the peptide sequence having cell survival properties comprises at least one XIAP fragment having cell survival properties selected which is the BIR2 domain with the linker domain between the BIR1 and BIR2 domains.

The invention concerns more particularly a pharmaceutical composition as defined above, wherein the peptide sequence having cell survival properties is the domain represented by SEQ ID NO: 8 or represented by SEQ ID NO: 44.

The invention also concerns more particularly a pharmaceutical composition as defined above, wherein the fusion protein is PTD-BIR2 represented by SEQ ID NO: 24 or PTD-BIR2 represented by SEQ ID NO:46.

The invention also relates more particularly to a pharmaceutical composition as defined above, wherein the peptide sequence having cell survival properties comprises at least one XIAP fragment having cell survival which is the BIR3-RING domain or the BIR3-RING linker domain.

The invention concerns more particularly a pharmaceutical composition as defined above, wherein the peptide sequence having cell survival properties is selected from the list comprising:
the BIR3-RING domain represented by SEQ ID NO: 10,
the BIR3-RING domain represented by SEQ ID NO: 12,
the BIR3-RING linker domain represented by SEQ ID NO:36,
the BIR3-RING linker domain represented by SEQ ID NO:38.

The invention also concerns more particularly a pharmaceutical composition as defined above, wherein the fusion protein is selected from the list comprising:
PTD-BIR3-RING represented by SEQ ID NO: 26, corresponding to the fusion of SEQ ID NO: 2 and SEQ ID NO: 10,
PTD-BIR3-RING represented by SEQ ID NO: 28, corresponding to the fusion of SEQ ID NO: 2 and SEQ ID NO: 12,
PTD-(BIR3-RING linker) represented by SEQ ID NO: 40, corresponding to the fusion of SEQ ID NO: 2 and SEQ ID NO: 36,
PTD-(BIR3-RING linker) represented by SEQ ID NO: 42, corresponding to the fusion of SEQ ID NO: 2 and SEQ ID NO: 38.

The invention also relates to a pharmaceutical composition comprising as active substance a nucleic acid coding for a fusion protein as defined above, in association with a pharmaceutically acceptable carrier.

The invention also relates to a fusion protein constituted of:
a fusion protein comprising or constituted of:
   at least one peptide sequence having membrane transducing properties, and
   at least one peptide sequence having cell survival properties; or
a homologous protein derived from said fusion protein by insertion, deletion, or substitution of at least one amino acid, provided that said homologous protein presents an identity percentage of at least 85%, and preferably of at least 89%, with respect to said fusion protein and presents membrane transducing and cell survival properties.

The invention concerns more particularly a fusion protein as defined above, wherein the peptide sequence having membrane transducing properties is the protein transduction domain (PTD) of the HIV TAT protein.

The invention relates more particularly to a fusion protein as defined above, wherein the peptide sequence having membrane transducing properties is the protein transduction domain (PTD) of the HIV TAT protein represented by SEQ ID NO: 2.

The invention concerns more particularly a fusion protein as defined above, wherein the peptide sequence having cell survival properties is selected from the list comprising:
the X chromosome-linked apoptosis inhibitor protein (XIAP) or fragments thereof having cell survival properties, such as the BIR2, BIR3-RING, or the BIR3-RING linker domains of XIAP, or
the Flice inhibitory protein (FLIP) or fragments thereof having cell survival properties.

The invention concerns more particularly a fusion protein as defined above, wherein the peptide sequence having cell survival properties is selected from the list comprising:
the XIAP represented by SEQ ID NO: 4 (rat),
the XIAP represented by SEQ ID NO: 6 (human),
the BIR2 domain represented by SEQ ID NO: 8 (rat),
the BIR2 domain represented by SEQ ID NO: 44 (human),
the BIR3-RING domain represented by SEQ ID NO: 10 (rat),
the BIR3-RING domain represented by SEQ ID NO: 12 (human),
the BIR3-RING linker domain represented by SEQ ID NO: 36 (rat),
the BIR3-RING linker domain represented by SEQ ID NO: 38 (human),
the FLIP represented by SEQ ID NO: 14 (FlipL) (mouse),
the FLIP represented by SEQ ID NO: 16 (FlipS) (mouse),
the FLIP represented by SEQ ID NO: 18 (Flip control) (mouse).

The invention relates more particularly to a fusion protein as defined above, wherein the fusion protein is selected from the list comprising:
PTD-XIAP represented by SEQ ID NO: 20, corresponding to the fusion of SEQ ID NO:2 and SEQ ID NO: 4,
PTD-XIAP represented by SEQ ID NO: 22, corresponding to the fusion of SEQ ID NO: 2 and SEQ ID NO: 6,
PTD-BIR2 represented by SEQ ID NO: 24, corresponding to the fusion of SEQ ID NO: 2 and SEQ ID NO: 8,
PTD-BIR2 represented by SEQ ID NO: 46, corresponding to the fusion of SEQ ID NO: 2 and SEQ ID NO: 44,
PTD-BIR3-RING represented by SEQ ID NO: 26, corresponding to the fusion of SEQ ID NO: 2 and SEQ ID NO: 10,
PTD-BIR3-RING represented by SEQ ID NO: 28, corresponding to the fusion of SEQ ID NO: 2 and SEQ ID NO: 12,
PTD-(BIR3-RING linker) represented by SEQ ID NO: 40, corresponding to the fusion of SEQ ID NO: 2 and SEQ ID NO: 36,
PTD-(BIR3-RING linker) represented by SEQ ID NO: 42, corresponding to the fusion of SEQ ID NO: 2 and SEQ ID NO: 38,
PTD-FLIP represented by SEQ ID NO: 30, corresponding to the fusion of SEQ ID NO: 2 and SEQ ID NO: 14,
PTD-FLIP represented by SEQ ID NO: 32, corresponding to the fusion of SEQ ID NO: 2 and SEQ ID NO: 16,
PTD-FLIP represented by SEQ ID NO: 34, corresponding to the fusion of SEQ ID NO: 2 and SEQ ID NO: 18.

The invention also concerns a fusion protein constituted of:
a fusion protein comprising or constituted of:
   at least one peptide sequence having membrane transducing properties, and
   at least one peptide sequence having cell survival properties selected from the list comprising:
      fragments of the X chromosome-linked apoptosis inhibitor protein (XIAP) having cell survival properties, or
      fragments of the Flice inhibitory protein (FLIP) having cell survival properties,
a homologous protein derived from said fusion protein by insertion, deletion, or substitution of at least one amino acid, provided that said homologous protein presents an identity percentage of at least 85%, and preferably of at least 89%, with respect to said fusion protein and presents membrane transducing and cell survival properties,
   provided that said fusion protein or said homologous protein does not comprise a whole XIAP sequence.

The invention relates more particularly to a fusion protein as defined above, wherein the peptide sequence having cell survival properties comprises at least one XIAP fragment having cell survival properties selected from the list comprising:
the BIR2 domain, or
the BIR3-RING domain, or
the BIR3-RING linker domain.

The invention also relates more particularly to a fusion protein as defined above,

wherein the peptide sequence having cell survival properties comprises at least one XIAP fragment having cell survival properties selected which is the BIR2 domain or linker between the BIR1 and BIR2 domains.

The invention concerns more particularly a fusion protein as defined above, wherein the peptide sequence having cell survival properties is the domain represented by SEQ ID NO: 8 or represented by SEQ ID NO: 44.

The invention also concerns more particularly a fusion protein as defined above, wherein the fusion protein is PTD-BIR2 represented by SEQ ID NO: 24 or PTD-BIR2 represented by SEQ ID NO:46.

The invention also relates more particularly to a fusion protein as defined above, wherein the peptide sequence having cell survival properties comprises at least one XIAP fragment having cell survival which is the BIR3-RING domain or the BIR3-RING linker domain.

The invention concerns more particularly a fusion protein as defined above, wherein the peptide sequence having cell survival properties is selected from the list comprising:
the BIR3-RING domain represented by SEQ ID NO: 10,
the BIR3-RING domain represented by SEQ ID NO: 12,
the BIR3-RING linker domain represented by SEQ ID NO:36,
the BIR3-RING linker domain represented by SEQ ID NO:38.

The invention also concerns more particularly a fusion protein as defined above, wherein the fusion protein is selected from the list comprising:
PTD-BIR3-RING represented by SEQ ID NO: 26, corresponding to the fusion of SEQ ID NO: 2 and SEQ ID NO: 10,
PTD-BIR3-RING represented by SEQ ID NO: 28, corresponding to the fusion of SEQ ID NO: 2 and SEQ ID NO: 12,
PTD-(BIR3-RING linker) represented by SEQ ID NO: 40, corresponding to the fusion of SEQ ID NO: 2 and SEQ ID NO: 36,
PTD-(BIR3-RING linker) represented by SEQ ID NO: 42, corresponding to the fusion of SEQ ID NO: 2 and SEQ ID NO: 38.

The invention also relates to a nucleic acid coding for a fusion protein as defined above.

The invention concerns more particularly a nucleic acid as defined above, represented by SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO:45, SEQ ID NO: 39, or SEQ ID NO: 41.

The invention also concerns more particularly a nucleic acid as defined above, represented by SEQ ID NO: 23, SEQ ID NO: 29, SEQ ID NO: 31, SEQ ID NO: 33, SEQ ID NO:45, SEQ ID NO: 39, or SEQ ID NO: 41.

The invention also relates to an eukaryotic or prokaryotic expression vector comprising a nucleic acid as defined above in association with the genetic elements necessary for its expression.

The invention concerns more particularly an eukaryotic or prokaryotic cell comprising a vector as defined above.

The invention also concerns a process for preparing a fusion protein as defined above, comprising:
the in vitro culture of transformed eukaryotic or prokaryotic cell comprising a vector as defined above,
the recovery, and if necessary the purification, of the fusion protein produced in said transformed cells.

### DESCRIPTION OF THE FIGURES

FIG. 1A, FIG. 1B and FIG. 1C

### Structure, Purity, Integrity and Tissue Transduction of PTD-XIAP

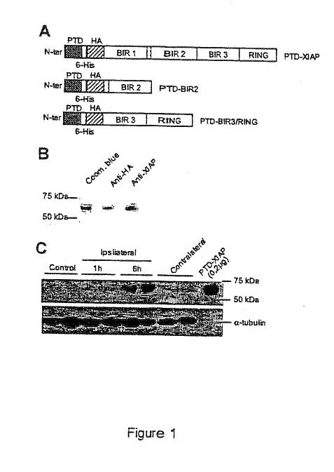

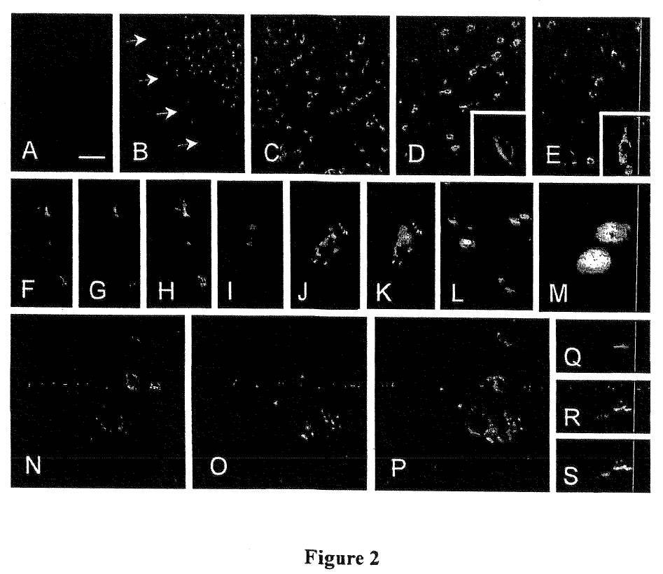

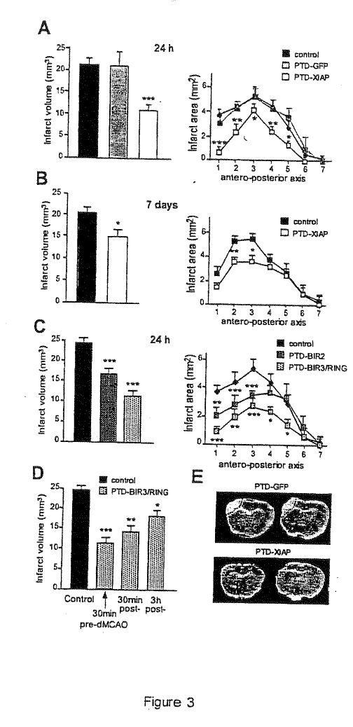

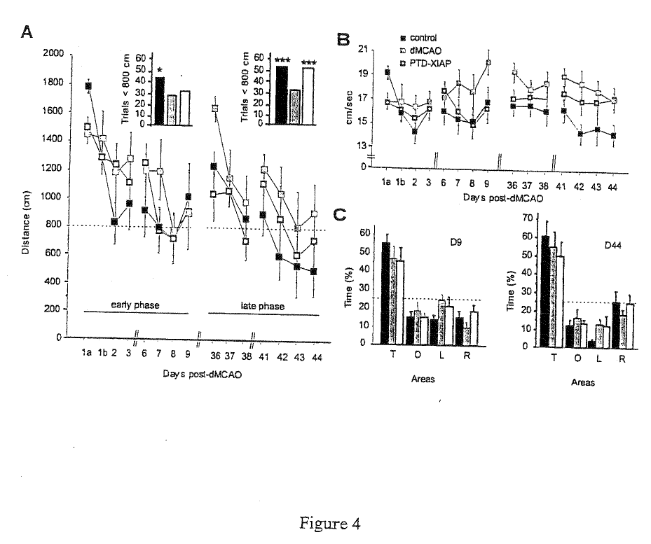

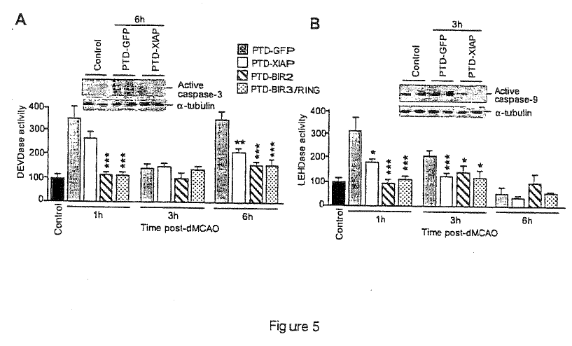

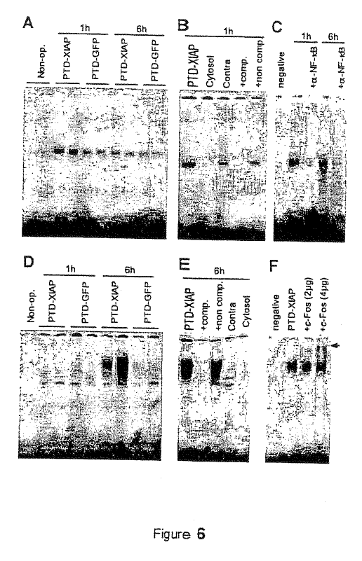

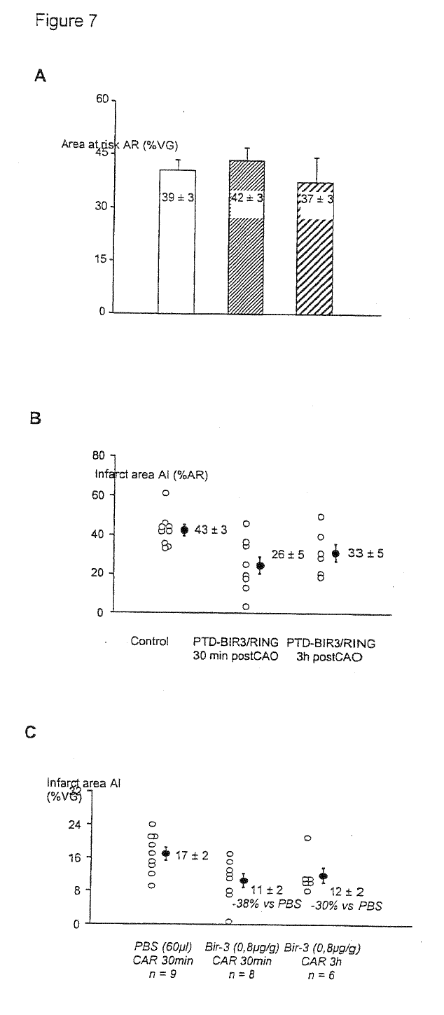

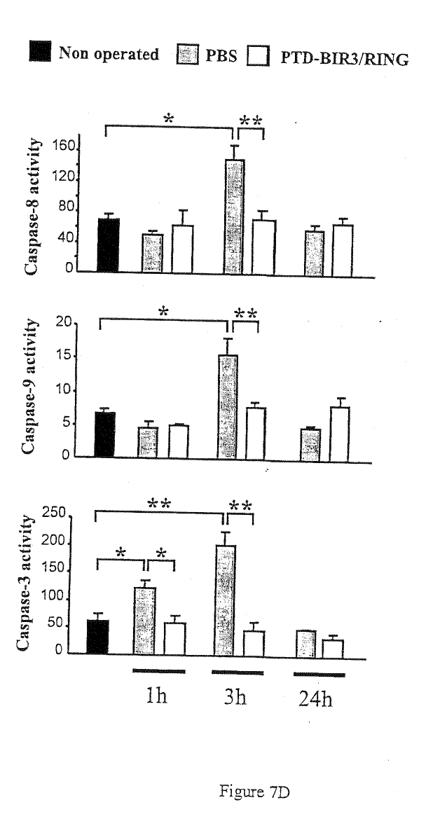

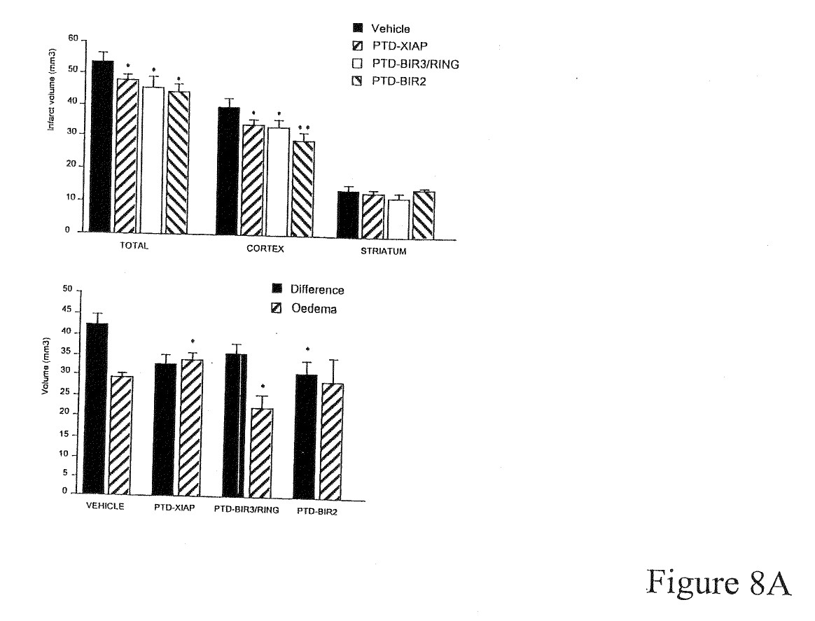

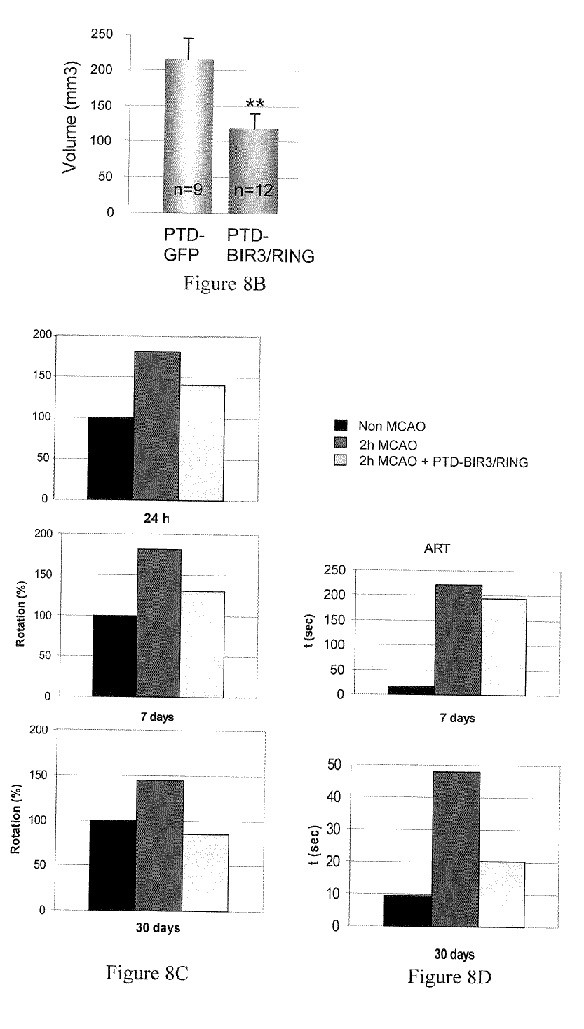

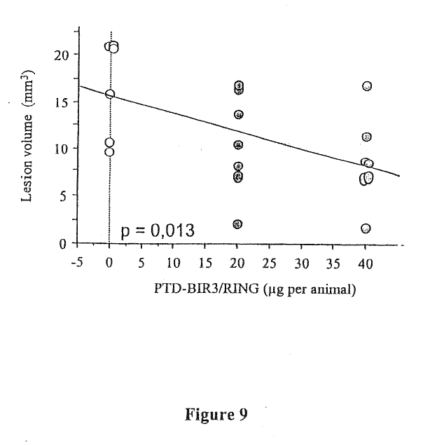

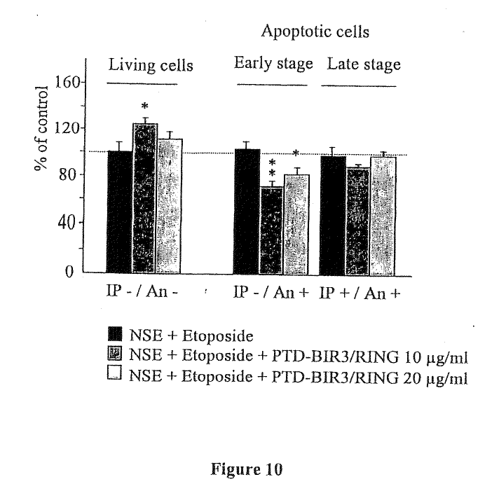

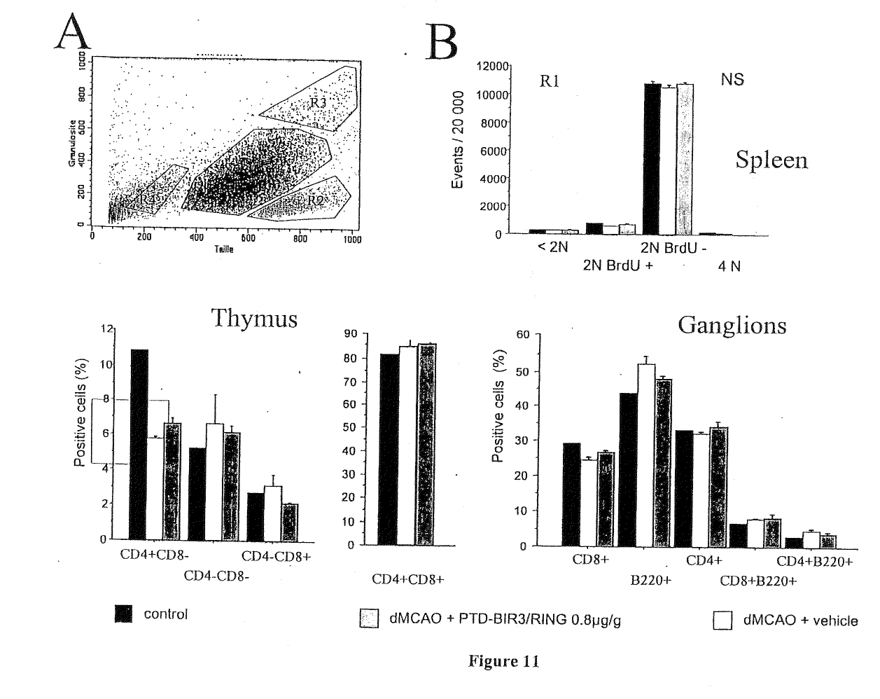

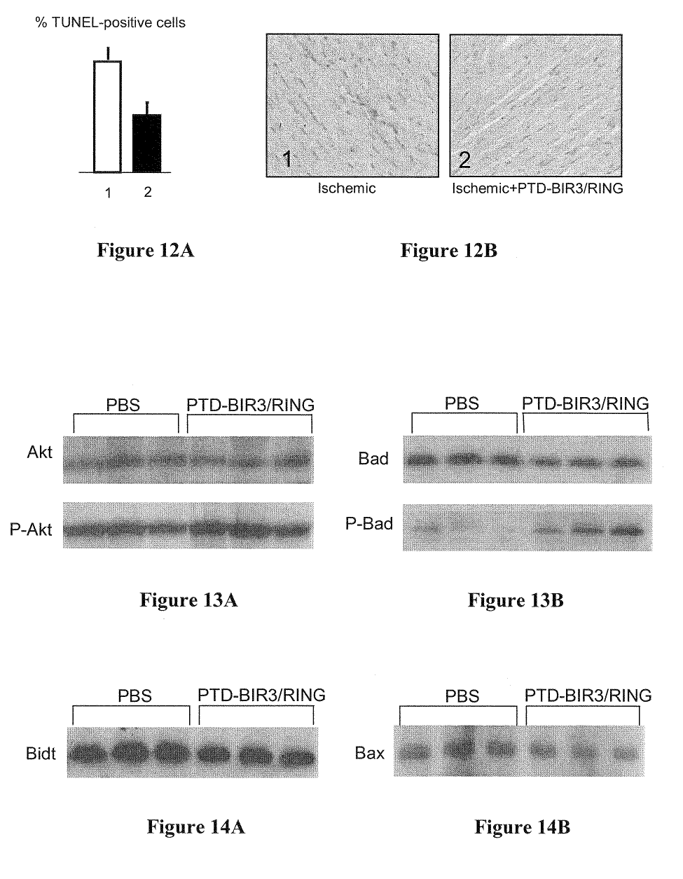

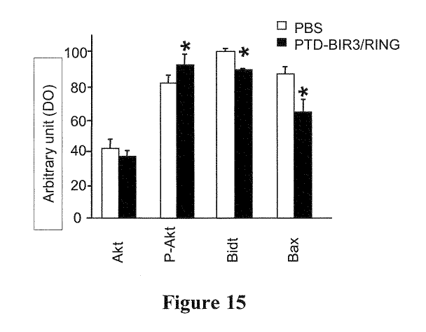

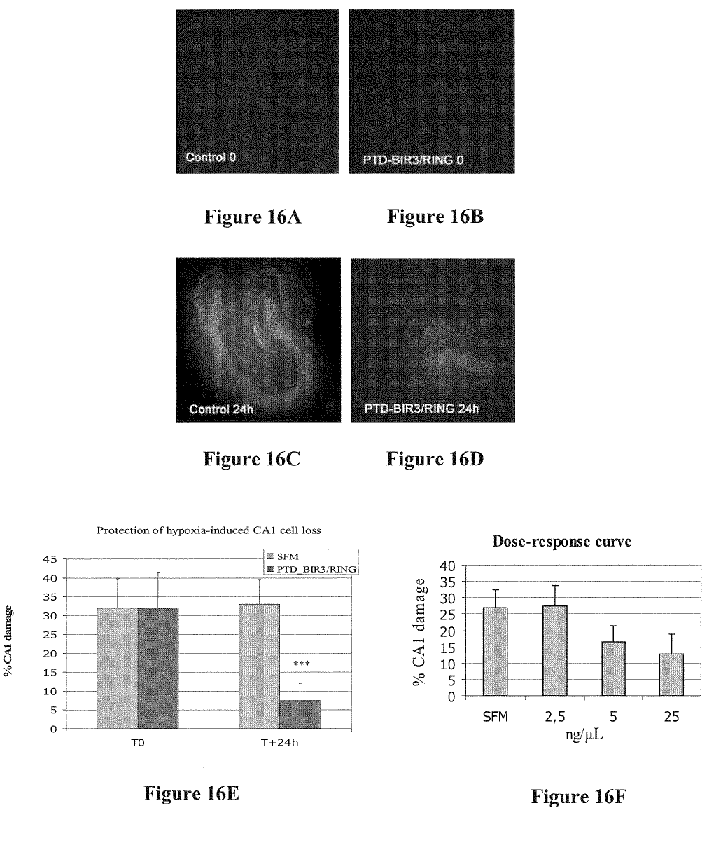

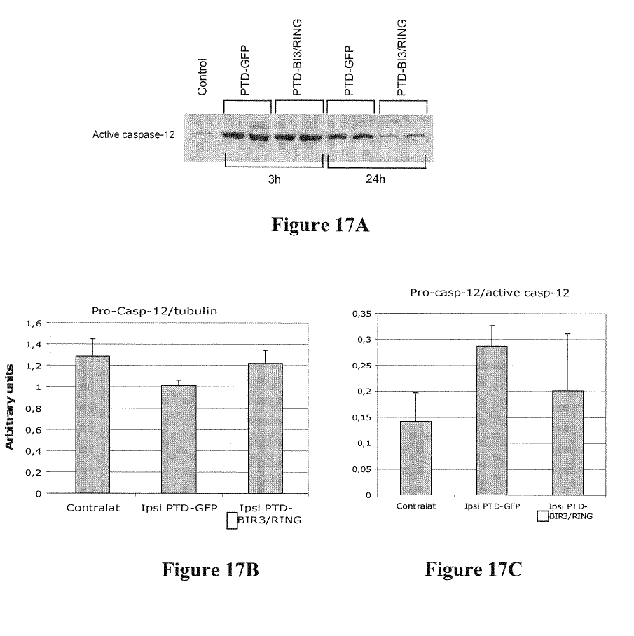

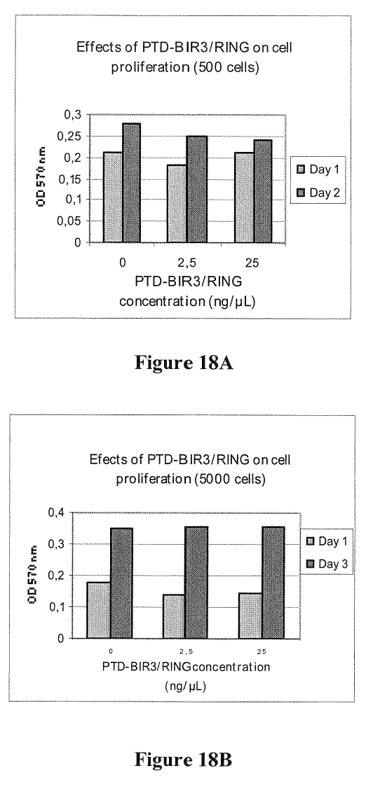

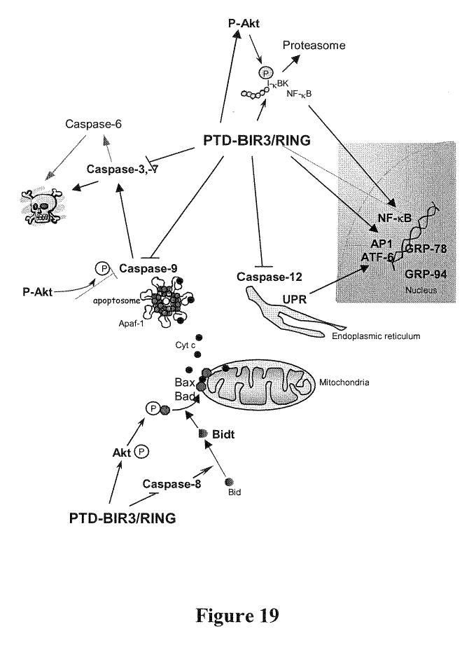

FIG. 1A: PTD fusion protein contains the HA tag, to distinguish it from endogenous proteins, and 6-His used for purification.

FIG. 1B: Coomassie blue stained acrylamide gel after electrophoresis of 2 μg PTD-XIAP to assess the integrity of the protein, recognized as a single band by both anti-HA and anti-XIAP antibodies.

FIG. 1C: Western blot (anti-HA antibodies) of PTD-XIAP in cortical tissues of non operated mice (Control), and 1 h and 6 h post-pMCAO in cortical tissues ipsilateral and contralateral to the occlusion. Each lane corresponds to a different animal (n=3 per group, topical administration). The purified protein (PTD-XIAP) in the last lane is shown for comparison. α-tubulin was used as an internal loading control.

FIG. 2A, FIG. 2B, FIG. 2C, FIG. 2D, FIG. 2E, FIG. 2F, FIG. 2G, FIG. 2H, FIG. 2J, FIG. 2K, FIG. 2L, FIG. 2M, FIG. 2N, FIG. 2O, FIG. 2P, FIG. 2Q, FIG. 2R and FIG. 2S

Transduction of PTD-XIAP into the Ischemic Brain 6 h after Topical Application of 20 μg

No HA is observed in the contralateral cortex of ischemic mice (FIG. 2A), or (FIG. 2B) in the tissue surrounding the arterial territory (arrows indicate the border between ischemic and non-ischemic tissues), in contrast to the infarcted area (FIG. 2C). Most neurons labelled with NeuN were labelled with HA (FIG. 2D).

FIGS. 2E-2G: Double immunofluorescence shows co-localization of PTD-XIAP (HA staining) with glial GFAP (FIGS. 2F-2G), and neuronal NeuN (FIGS. 2H-2J) markers, indicating transduction into both cell types. (FIGS. 2K-2M) XIAP and HA antibodies reveal the intracellular distribution of endogenous and exogenous XIAP. Bar=FIGS. 2A-2C: 150; FIG. 2D: 58 μm; FIGS. 2E-2G, 60 μm; FIGS. 2H-2J: 25 μm; FIGS. 2K-2M: 18 μm.

FIG. 3A, FIG. 3B and FIG. 3C

### Protection Against Ischemic Damage by PTD-XIAP Applied Topically

FIGS. 3A-3B (left panels): Protection 24 h (FIG. 3A), and 7 d. (FIG. 3B) post-pMCAO by the full-length protein versus application of physiological serum-soaked gel (control), or PTD-GFP. FIGS. 3A-3B (right panels) illustrate infarct sizes in the 7 lesioned brain levels. Protection by PTD-XIAP is more significant at frontal levels at 24 h (FIG. 3A), and persists below gel location at 7 d. FIG. 3C: Protection by PTDBIR2 and PTD-BIR3/RING at 24 h (left), and rostro-caudal distribution of infarct sizes (right).

FIG. 3D: Effects of a single injection of PTD-BIR3/RING performed 30 min before, or 30 min and 3 h after dMCAO. Data are mean T SEM; n=5-8 per group. *P<0.05, **P<0.01, ***P<0.001, versus control (ANOVA and Student's t test).

FIG. 3E: Two representative photographs of TTC-stained coronal sections from pMCAO mice at 24 h. The dotted black lines illustrate the infarct border.

FIG. 4A, FIG. 4B and FIG. 4C

Performance in the Morris Water Maze (n=7 Per Group).

Distance to the platform (FIG. 4A), and swim speed (FIG. 4B). Inserts in FIG. 4A indicate the number of trials to find the platform in less than 8 m. FIG. 4C: Results of the two probe trials. T: target quadrant; O: opposite quadrant, L: left quadrant, R: right quadrant. *p<0.05, ***p<0.001, versus dMCAO (two-way ANOVA; PLSD Fisher's post-hoc test).

FIG. 5A and FIG. 5B

Activities of caspase-3 (FIG. 5A) and caspase-9 (FIG. 5B), expressed in μmoles of AFC released/h.mg protein, evaluated 1 h, 3 h, and 6 h after pMCAO (n=6-8). Control: non-operated mice. Top panels show examples of Western blot analysis of active caspase-9 and caspase-3, 3 h and 6 h post-pMCAO, respectively, in the left cortex of non-operated (Control), and ischemic PTD-GFP- and PTD-XIAP-treated mice. Each lane corresponds to a different animal (n=3 per group). Data are mean±SEM, expressed as percentiles of control. * p<0.05; ** p<0.01;*** p<0.001 versus control (one-way ANOVA; Fischer's post-hoc test).

FIG. 6A, FIG. 6B, FIG. 6C, FIG. 6D, FIG. 6E and FIG. 6F

EMSA showing NF-κB (FIGS. 6A-6C) and AP1 (FIGS. 6D-6F) DNA binding activities in non-operated (Non-op.), PTD-XIAP-, and PTD-GFP-treated mice 1 h and 6 h after pMCAO. Each lane corresponds to a different animal (n=3 per group).

FIG. 6B: Competition assay performed on PTD-XIAP-treated mice 1 h after pMCAO, using 100 fold-excess unlabeled NF-κB probe (+comp.) and the non-specific competitor TFIID (+non comp.). DNA binding in the side contralateral to ischemia (Contra), in cytosolic protein fractions (Cytosol) obtained from PTD-XIAP-treated animals.

FIG. 6C: NF-κB activity in PTD-XIAP-treated extracts incubated with (+α-NF-κB), or without, anti-NF-κB antibodies, or in incubation medium devoid of protein (negative).

FIG. 6D: API DNA binding activity, 1 h and 6 h post-pMCAO in PTD-XIAP- versus PTD-GFP-treated animals.

FIG. 6E: Competition assay performed on PTD-XIAP-treated mice 6 h post-pMCAO, using 100-fold excess unlabeled API probe (+comp.), or a non-specific competitor OCT1 (+non comp.), in nuclear proteins from contralateral cortex (Contra), and in cytosolic extracts (Cytosol).

FIG. 6F: Supershift of AP1 binding (arrowhead) by specific antibodies against c-Fos (2 μg or 4 μg) 6 h post-pMCAO. (Negative): incubation medium without proteins.

FIG. 7A, FIG. 7B, FIG. 7C, FIG. 7D

### Protection by PTD-BIR3/RING in a Murine Model of Ischemic Cardiopathy.

Ischemia is induced in mice by the insertion into the myocardium of an occlusive thread that encircles tightly the coronary artery. Coronary artery occlusion (CAO) is performed for 30 min. Mice receive an injection of buffer solution (PBS) or of PTD-BIR3/RING (0.8 mg/kg) into the carotid artery, 30 min or 3 h after reperfusion (CAR, coronary artery reperfusion). Animals are sacrificed and the volumes of the infarcted area (IA) and of the area at risk (AR) are measured on each animal.

FIG. 7A: Average volume of the area at risk in normal mice (white), mice with ischemia treated with PTD-BIR3/RING 30 min (thin hatches), and 3 h (big hatches) after reperfusion.

FIG. 7B: Average volume of the infarct in normal mice (control), mice with ischemia treated with PTD-BIR3/RING at 30 min and 3 h after reperfusion (PTD-BIR3/RING 3 h post-CAR).

FIG. 7C: Percentage of the IA (Infarct Area) over the total volume of the left ventricle in normal mice (PBS (60 μL) CAR 30 min), mice after ischemia and treated with PTD-BIR3/RING at 30 min (Bir-3 (0.8 μg/g) CAR 30 min), and 3 h after reperfusion (Bir-3 (0.8 μg/g) CAR 3 h).

FIG. 7D: Inhibition of caspase-3, caspase-8, and caspase-9 by PTD-BIR3/RING in the cardiac muscle 1 h, 3 h and 24 h (n=4-6) after reperfusion following a 30 min CAO. The protein or its vehicle solution (PBS) were injected 30 min after reperfusion into the jugular vein.

FIG. 8A, FIG. 8B, FIG. 8C, FIG. 8D

The Effects of a Single Intravenous Injection of PTD-BIR3/Ring on Infarct Volume 24 H after 2 h of MCAO in Rats, and on Long-Term Functional Deficits.

FIG. 8A: Administration of PTD-BIR3/RING (0.12 mg/kg) 30 min before occlusion results in significant protection of the volume of infarcted tissue and of the oedema, indicating that penetration of the protein is efficient when the blood brain barrier is intact.

FIG. 8B: Administration of the protein (0.24 mg/kg) at the moment of reperfusion after 2 h MCAO results in significant decrease of lesion volumes (** P<0.001) compared to PTD-GFP injection.

FIG. 8C: Decrease of infarct size correlates with total reversal of ischemia-induced functional deficits on the apomorphin-induced rotation test after one month. A 23% and 32% reduction is observed after 24 h and 7 days, respectively. Data are % of values obtained in non-ischemic animals.

FIG. 8D: Recovery of motor functions are associated with long-lasting decreased impairment of sensori-motor functions assessed with the adhesive removal test. Latency to remove an adhesive on the left paw is reduced by 12% at 7 days and 58% after one month.

FIG. 9

Relationships Between the Dose of PTD-BIR3/RING and the Extent of Cerebral Protection after Permanent MCAO

Protection of lesion volumes induced by dMCAO in mice by 0.8 μg/g and 1.6 μg/g of PTD-BIR3/RING injected into the tail vein 30 min after arterial occlusion in mice.

FIG. 10

Protection of Neurospheres-Derived Cells from Etoposide-Induced Apoptosis by PTD-BIR3/RING.

Neurospheres are prepared from mouse E14 embryos according to Reynolds and Weiss (1992), and subjected to 100 μM etoposide for 24 hours. PTD-BBIR3/RING is applied for 4 h at 25 ng/μL before cell counting by fluorescence-activated cell counting with Annexin-V labeling.

FIG. 11A and FIG. 11B

### Lack of Modifications of Lymphocytic Populations

Effects of PTD-BIR3/RING administered iv (0.8 μg/g) 30 min after dMCAO in mice on lymphocytic populations by fluorescent-activated cell sorting. PTD-BIR3/RING used in concentrations efficient to reduce brain damage shows no adverse effects on lymphocytic populations.

FIG. 11A: Analysis of cells in the spleen reveals 4 populations, with a majority for the population R1.

FIG. 11B: No differences are observed after treatment of the animals with PTD-BIR3/RING in the 4 cell populations present in the spleen, nor in lymphocytes identified as CD4- or CD8-positive by immunofluorescence in lymph nodes and thymus.

FIG. 12A and FIG. 12B

Reduction of Apoptosis in Cardiomyocytes after Administration of PTD-BIR3/RING in cardiac ischemia

FIG. 12A: TUNEL labeling of myocardium after 30 min CAO in mice reveals apoptotic cells (1). Treatment with PTD-BIR3/RING significantly reduces the number of TUNEL-positive cardiomyocytes (2).

FIG. 12B: Histological section corresponding to the conditions 1 and 2 of FIG. 12A. Apoptotic cells appear in brown.

FIG. 13

### Administration of PTD-BIR3/RING Increases the Ratio of Phosphorylated Versus Non-Phosphorylated Forms of the Serine-Threonine Kinase Akt, and the Pro-Apoptotic Member of the Bcl2 Family Bad (Datta et al., 2002)

FIG. 13A: PTD-BIR3/RING induces no modifications of the non-phosphorylated form of Akt (upper panel), but significantly increases the phosphorylated form (lower panel).

FIG. 13B: Western blotting for the pro-apoptotic non phosphorylated form and inactive phosphorylated form of Bad. Administration of PTD-BIR3/RING significantly increases the phosphorylated form.

FIG. 14

### Administration of PTD-BIR3/RING Decreases Bax (Korsmeyer et al., 2000) and the Truncated Form of Bid (Reed et al., 2006).

FIG. 14A: Western blotting for the truncated form of Bid (Bidt) showing decreased contents in cardiac tissues of mice after administration of PTD-BIR3/RING 30 min after CAR.

FIG. 14B: Western blotting for Bax showing decreased contents in cardiac tissues of mice after administration of PTD-BIR3/RING 30 min after CAR.

FIG. 15

Quantification of the Modifications of Akt (Song et al., 2005), Phosphorylated Akt (P-Akt), Truncated Bid (Bidt) and Bax after Administration of PTD-BIR3/RING 30 min after Reperfusion Following 30 min of CAO.

PTD-BIR3/RING (0.8 mg/kg in 100 μl) or PBS (100 μl) was administered intravenously.

FIG. 16

### Analysis of the Neuroprotective Effect of PTD-BIR3/RING on Hypoxia-Induced Neuronal Death in Hippocampal Brain Slices

FIGS. 17A and 17B: Slices cultured in normal conditions, without (A) or with (B) PTD-BIR3/RING show no PI (propidium iodide) labeling at the time of hypoxia induction.

FIG. 17C: Twenty four hour later, strong incorporation of PI is observed in control slices, indicating massive cell death.

FIG. 17D: Addition of PTD-BIR3/RING dramatically reduces PI incorporation.

FIG. 17E: Quantification of the protective effect of PTD-BIR3/RING on hypoxia-induced cell death. ***p<0.0001.

FIG. 17F: Dose-response curve with increasing concentrations of PTD-BIR3/RING. Significant protection (p<0.005) is observed for doses of 5 ng/μL or more.

FIG. 17

### Intracellular Signaling of PTD-BIR3/RING: Inhibition of the Endoplasmic Reticulum-Related Death Pathway

FIG. 18A: Representative Western blot showing MCAO-induced modifications of the active form of caspase-12 at 3 and 24 hours. Control: normal mice; PTD-GFP: MCAO mice treated intravenously with 20 μg of PTD-GFP protein; PTD-BIR3/RING: MCAO mice treated intravenously with 20 μg of PTD-BIR3/RING.

FIGS. 18B and 18C: Quantification of Western blot results for caspase-12. Ratio of caspase-12 pro-form to tubulin contents (FIG. 18B) and to caspase-12 active form (FIG. 18C). Contralat: contralateral cortex, Ipsi PTD-GFP: ipsilateral cortex of PTD-GFP-treated mice mice; Ipsi PTD-BIR3/RING: ipsilateral cortex of PTD-BIR3/RING-treated mice.

FIG. 18

### Absence of Effect of PTD-BIR3/RING on Cell Proliferation In Vitro

Analysis of the proliferation of the ND7 cell line after administration of increasing doses of PTD-BIR3/RING over 2 (left) or 3 (right) days.

Two cell densities were used: 500 (FIG. 19A) and 5000 (FIG. 19B) cells. PTD-BIR3/RING induced no modifications of the growth curves.

FIG. 19

### A Summary of PTD-BIR3/RING Signaling Pathways

According to the results of the Inventors, PTD-BIR3/RING exerts its anti-ischemic effect through activation of survival pathways correlated with inhibition of pro-apoptotic pathways. Survival pathways include activation of Akt/PKB, activation of transcription factors (NFκB and API), and decrease of Bad contents. Inhibition of pro-apoptotic pathways includes caspase inhibition, phosphorylation of Bad, and reduction of the truncated form of Bid. Importantly, caspase inhibition is not restricted to the well known effects on caspase-3 and -9, but involves caspase-12, leading to reduction of endoplasmic reticulum stress-related mechanisms, and involves caspase-8, leading to reduction of the <<death receptor pathway>> of apoptosis.

### EXAMPLES

### Materials and Methods

Generation, production and purification of PTD-XIAPs. The cDNA encoding the entire reading frame of rat XIAP was isolated with PCR using the primers 5′-GGG CTC GAG ATG ACT TTT AAC AGT TTT GAA GG-3′ (sense) and 5′-GGG GAA TTC TTA AAA CAT AAA AAT TTT TTT GCT TG-3′ (antisense). Purified fragments were cloned into the XhoI/EcoRI sites of the pPTD-HA vector. Two additional constructs were cloned, encoding the BIR2 domain and its associated linker region to BIR1 (PTD-BIR2), or the BIR3/RING domains (PTD-BIR3/RING) (FIG. 1A). The integrity of the constructs was confirmed by DNA sequencing. Plasmids were expressed in E. coli strain BL21(DE3) pLysS (Stratagene, La Jolla, Calif., USA) as described (Dietz et al., 2002). Protein production was induced by addition of 500 μM isopropyl 1-thio-β-D-galactoside. Proteins were extracted in 8M urea HEPES buffer, and were purified using a Ni-NTA superflow agarose column (Qiagen, Hilden, Germany). Salt was removed by gel filtration on Sephadex G-25M (Amersham Pharmacia Biotech, Uppsala, Sweden), and proteins were collected in a solution containing phosphate buffered saline (PBS). A p-PTD-HA-green fluorescent protein (GFP) construct was used as control (kindly provided by SF. Dowdy; Schwarze et al., 1999). The molecular weight of purified proteins was verified by Coomassie blue staining and Western blotting. The final cDNA encoded a protein comprising a 6-histidine residues tag, the PTD (YGRKKRRQRRR), a hemagglutinin (HA) tag (YPYDVPDVA), and XIAP, BIR2, or BIR3/RING (FIG. 1A). The integrity of the construct was confirmed by DNA sequencing. Plasmid was expressed in E. coli strain BL21(DE3) pLysS (Stratagene, La Jolla, Calif., USA) as described (Dietz et al., 2002). Protein production was induced by addition of 500 μM isopropyl 1-thio-p-D-galactoside. Proteins were extracted in 8M urea HEPES buffer, and were purified using a Ni-NTA superflow agarose column (Qiagen, Hilden, Germany). Salt was removed by gel filtration on Sephadex G-25M (Amersham Pharmacia Biotech, Uppsala, Sweden), and proteins were collected in a solution containing phosphate buffered saline (PBS). A p-PTD-HA-green fluorescent protein (GFP) construct was used as control (provided by S F. Dowdy; Schwarze et al., 1999). The molecular weight of purified protein was verified by Coomassie blue staining and Western blotting (FIG. 1B).

The construct encoding the rat BIR2 domain (PTD-BIR2) has been realised by subcloning the full-length XIAP sequence with the following primers Forward: 5′-AGA AAT CAT TTT GCT CTT GAC AGG-3′, Reward: 5′-C AAG CAA AAA ATT TTT ATG TTT TAA-3′. Purified fragments were cloned into the XhoI/EcoRI sites of the pPTD-HA vector. The construct encoding the rat BIR3/RING domain (PTD-BIR3/RING) has been realised by subcloning the full-length XIAP sequence with the following primers Forward: 5′-ATG GCA GAA TAT GAC GCA CGG-3′, Reward: 5′-C AAG CAA AAA ATT TTT ATG TTT TAA-3′. Purified fragments were cloned into the XhoI/EcoRI sites of the pPTD-HA vector. The construct encoding the human BIR3/RING domain (hPTD-BIR3/RING) has been realised by subcloning of the full-length XIAP with the following primers Forward: 5′-ATG GCA GAT TAT GAA GCA CGG-3′, Reward: 5′-C AAG CAA AAA ATT TTT ATG TCT TAA-3′. Purified fragments were cloned into the XhoI/EcoRI sites of the pPTD-HA vector.

Permanent focal ischemia in mice. All the studies with mice were conducted in accordance with the recommendations of the European community (86/609/EEC) for care and use of laboratory animals. Eight week-old male C57B1/6 mice (20-22 g Janvier, Le Genest-St-Isle, France) were anesthetized with 4% halothane for induction and 2% halothane in 70% N₂O and 30% O₂ during surgical procedures. PMCAO (Permanent Middle Cerebral Artery Occlusion) was performed according to Gotti et al. (1990) by electrocoagulation (Aesculap^{R} GN 60, Tuttlingen, Germany) and section of the MCA to avoid reperfusion. Rectal temperature was maintained between 36.7 and 37.5° C. by using a homeothermic blanket (Harvard Apparatus). Arterial blood pressure and blood gas composition were monitored by blood sampling from the left femoral artery. Arterial blood samples were analyzed for pH, arterial oxygen pressure and partial pressure of carbon dioxide (Corning, CIBA-Corning Diagnostics).

Transient focal ischemia in rats. Male adult (220-250 g) Sprague-Dawley rats are anesthetized and subjected to tMCAO (Transient Middle Cerebral Artery Occlusion) by insertion of a monofilament nylon thread into the internal artery up to the branching-off point of the MCA. Regional cerebral blood flow is monitored with a Laser Doppler (Optronix). The thread is removed after 2 h of occlusion. Rectal temperature is maintained between 26.7 and 37.5° C. by using a homeothermic blanket (Harvard Apparatus).

Protein administration was performed either by application of a 0.5 mm³ piece of gel foam containing 20 μg/100 μL of PTD-XIAP, or PTD-GFP, into the craniotomy hole performed for electrocoagulation of the artery, or by intravenous administration.

Measurement of infarct volume. Mice were sacrificed 24 h or 7 d after pMCAO. Brains were cut into 8 coronal 1 mm slices (Mouse Brain Slicer Coronal matrix; Harvard Apparatus, Holliston, Mass.). Sections were stained with 3% 2,3,5-triphenyltetrazolium chloride (TTC), and volumes of infarcted tissue were reconstructed with the KS400 software (Zeiss, Oberkocken, Germany) by subtracting the healthy areas of the lesioned cortex from identical areas in the contralateral cortex.

Western blot analysis. Proteins were extracted from contra- and ipsi-lateral cortices 1 h and 6 h after pMCAO (n=5 per group) in cold Tris buffer (50 mM Tris pH 7.4, 150 mM NaCl, 1 mM EDTA, 1 mM PMSF, and 5 t/ml protease inhibitor cocktail (P-8344, Sigma). Proteins were separated on 12-15% SDS-polyacrylamide gels using 30-50 μg proteins per lane. Immunoblots were processed as previously described (Benchoua et al, 2001) with mouse monoclonal anti-HA (clone 12CA5, Roche Mol. Bioch, Indianapolis, Ind., 1:500) or anti-tubulin (T5168, Sigma, France) antibodies, and rabbit polyclonal anti-XIAP (clone 2042, Cell Signaling Tech., Beverly, Mass., 1:500), anti-cleaved caspase-3 (R&D Systems, Minneapolis, Minn., 1:200), and anti-cleaved caspase-9 (AAP109, Stressgen Biotech., 1:200) antibodies. Bands were visualized with enhanced chemiluminescence (ECL+Detection System, Amersham Biosciences, Freiburg, Germany).

Immunohistochemical analysis was performed according to Benchoua et al. (2001) on cryostat sections obtained after fixation of the brain by intracardiac perfusion of 4% paraformaldehyde (PFA) in 0.1 M phosphate buffer (PB, pH 7.4) 1 h, 3 h, 6 h and 24 h after pMCAO (n=3 per group). Free-floating sections were incubated overnight at 4° C. with mouse monoclonal anti-HA (clone 12CA5, Roche Mol. Bioch., 1:200), rabbit anti-GFAP (Z0334, Dako, 1:1000), anti-NeuN (Boehringer-Mannheim, 1/500), or anti-XIAP (#2042, Cell Signaling Technology, 1:200) antibodies. Antigens were revealed with the Tyramide Amplification System (NEN, Boston, Mass.) with fluorescein or cyanin-3 as fluorophore. For double labeling, sections were processed for HA staining and then washed in acidic glycine buffer (0.1 M, pH 3.34) for 10 min (Nakane, 1968). This procedure does not detach former precipitates obtained with the amplification system and allows double fluorescent analysis. Sections were then reprobed with anti-GFAP, anti-NeuN, or anti-XIAP antibodies. Sections were examined with a Zeiss Axioplan II microscope equipped with a Coolsnap digital camera (Photometrics), or a Zeiss LSM confocal microscope (Zeiss), and the AxioVision software (Zeiss).

Behavioral testing. Spatial learning was assessed using the Morris water maze. An “early phase” training session was performed at days (D)1-3 and D6-9 post-occlusion, followed by a probe trial to evaluate the search strategy (D9). The probe trial quantifies the time spent in the four quadrants of the pool, i.e. the quadrant containing the platform (T), and the opposite (O), left (L) and right (R) quadrants. A second training session, or “late phase”, was conducted at D36-38 and D41-44, ending with a second probe trial (D44). Swimming performance was tracked and analysed using the View Point tracking system. Latency, inactivity, swim speed and path-length were measured for place training. We also measured the number of successful trials to reach the platform in less than 8 m, which is the maximal distance indicating acquisition of the strategy in our experimental paradigm.

Apomorphin-induced rotation: spontaneous preferential rotation is monitored for each rats the day before surgery.

Caspase activity assay. Proteins were extracted from contra- and ipsilateral cortices 1 h, 3 h, 6 h and 24 h after pMCAO (n=5 per group). Tissues were homogenized in cold buffer containing 25 mM HEPES pH 7.5; 5 mM MgCl₂; 2 mM EDTA; 0.1% Triton x×100; 2 mM dithiothreitol (DTT); 1 mM PMSF; 5 μl/ml protease cocktail inhibitor. Homogenates were centrifuged at 13000 g for 30 min at 4° C., and supernatants were collected. Proteins (50 μg) were incubated in caspase assay buffer (50 mM HEPES pH 7.4; 100 mM NaCl; 1 mM EDTA; 10 mM DTT) and the enzymatic reaction was started by addition of 0.2 mM fluorogenic substrate (Ac-DEVD-AFC or Ac-LEHD-AFC, Biomol Res. Labs.). Fluorescent arbitrary units were converted into μmoles of AFC/h.mg protein using a standard curve of free AFC (A8401, Sigma).

Electrophoretic mobility-shift assay (EMSA). Nuclear proteins were extracted from non-operated, PTD-GFP- and PTD-XIAP-treated mice sacrificed at 1 h or 6 h after pMCAO (n=3 per group), using NE-PER™ Nuclear and Cytoplasmic Extraction reagents (Pierce, Ill., USA), according to manufacturer instructions. Gel-shift assay was performed using Promega's DNA-binding protein detection system (E3050, E3300), as recommended by the manufacturer. Double-stranded oligonucleotide containing the consensus binding sequences of NF-κB (5′-AGT TGA GGG GAC TTT CCC AGG C-3′) and API (5′-CGC TTG ATG AGT CAG CCG GAA-3′) were radiolabeled with [γ-³²P]deoxy ATP 3000 Ci/mmol; Amersham Biosci., France) by T4 kinase (Promega, France), and were purified through G-25 spin column (Amersham Pharmacia Biotech., France). Ten μg nuclear proteins were incubated for 10 min at room temperature (RT) in 10 μl reaction containing 20% glycerol, 5 mM MgCl₂, 2.5 mM EDTA, 2.5 mM DTT, 250 mM NaCl, 50 mM Tris (pH 7.5), and 0.25 mg/ml poly(dI-dC). The binding reaction was initiated by adding 70 fmol of labeled probe. Protein-DNA complexes were resolved by electrophoresis through 4% native polyacrylamide gels at 4° C. in running buffer containing 45 mM Tris borate, 1 mM EDTA, pH 8. Gels were dried and exposed to Biomax MS films (Kodak, N.Y.) at −80° C. Adding 50 or 100-fold excess of unlabeled competitor before adding the radioprobe determined the specificity of DNA binding. Consensus sequences of OCT-1 (5-TGT CGA ATG CAA ATC ACT AGA A-3′) and TFIID (5′-GCA GAG CAT ATA AGG TGA GGT AGG A-3′) were used as unlabeled non-specific competitors for AP1 and NF-κB assays, respectively. Cytosolic extracts were used as internal control of the purification of cytoplasmic and nuclear proteins extracts.

For gel supershift assays, 10 μg of nuclear extracts were incubated with 2-4 μg anti-c-Fos or -NF-κB antibodies (SC-52× and SC-7178, respectively, Santa Cruz Biotech., CA) before adding the radiolabeled probe before EMSA. SC-7178 antibodies are raised against the DNA binding domain of NF-κB; their addition to the incubation medium therefore impairs NF-κB DNA binding, which results in a decrease of the specific band rather than in a mobility shift of the probe/protein complex on the gel.

Statistical analysis. Data are expressed as mean±S.E.M. One-way ANOVA was used to compare intergroup differences for protein contents or protease activities, followed with the appropriate post-hoc testing, as indicated in the figure legends. Differences were assumed statistically significant for p values <0.05.

### Results

### Example 1

### XIAP Fusion Proteins Against Cerebral and Cardiac Ischemia XIAP Fusion Proteins Efficiently Transduce Brain Cells In Vivo

The integrity of the construct was confirmed by Western blotting for XIAP or the HA tag (FIG. 1B). No PTD-XIAP was detected in brain regions after iv injection, or in the contralateral side of treated mice after topical administration (FIG. 1C). In contrast, PTD-XIAP was present in the lesioned area 1 h after application over the cortex and had conspicuously increased at 6 h post-occlusion (FIG. 1C). Immunoblot data were confirmed by HA-immunohistochemistry. While no HA-positive cells were detected in the cortex contralateral to the lesion (FIG. 2A), or in mice that had received a PTD-GFP-soaked gel (not shown), PTD-XIAP was present in cortical cells 1 h after its application (FIG. 2B). In contrast to PTD-XIAP, the both PTD-BIR2 and PTD-BIR3/RING were observed in the lesion after iv administration. All proteins were observed in GFAP-labeled astrocytes (FIGS. 2E-2G), and in NeuN-labeled neurons (FIGS. 2H-2K). Although this was not quantified, the great majority of NeuN-positive cells contained HA (FIG. 2K). HA-IR in PTD-XIAP-treated animals was punctate (FIGS. 2I, 2M-2O), and was restricted to the cytosolic compartment, including proximal neurites. Endogenous and exogenous XIAP were contained in different cytosolic compartments, as revealed by co-labeling with anti-XIAP and anti-HA antibodies (FIGS. 2M-2O). Treatment with BIR3/RING did not modify physiological parameters (Table 1).

TABLE 1
Physiological parameters during PTD-BIR3/RING treatment.

| | Group | 1 h* | 2 h* | 3 h* |
|---|---|---|---|---|
| pH | Vehicle | 7.332 ± 0.008 | 7.354 ± 0.013 | 7.365 ± 0.015 |
| | PTD-BIR3/ | 7.339 ± 0.004 | 7.337 ± 0.005 | 7.339 ± 0.009 |
| | RING | | | |
| pO2 | Vehicle | 94.10 ± 2.70 | 103.05 ± 6.05 | 102.85 ± 3.65 |
| | PTD-BIR3/ | 93.30 ± 4.69 | 97.52 ± 2.22 | 97.175 ± 3.46 |
| | RING | | | |
| pCO2 | Vehicle | 42.25 ± 1.85 | 34.90 ± 3.10 | 31.20 ± 1.50 |
| | PTD-BIR3/ | 41.95 ± 4.05 | 33.35 ± 3.15 | 30.86 ± 1.22 |
| | RING | | | |

| | 5 min before injection | 15 min after injection |
|---|---|---|
| Cardiac rhythm | 540 ± 40 | 510 ± 30 |
| MABP (mm Hg) | 96.5 ± 8.5 | 98.5 ± 6.5 |

*post-MCAO; MABP: mean arterial blood pressure.

XIAP Fusion Proteins Protect against dMCAO

Distal occlusion of the MCA (Middle Cerebral Artery) (dMCAO) induces a lesion restricted to the cortical territory of the artery (Guégan et al., 1998). Infarct volumes at 24 h were equivalent in animals untreated (control, 21.57±0.87 mm3) or treated with a PTD-GFP-soaked gel (21.49±2.71 mm3) (FIG. 3A, left panel), and in animals treated iv with XIAP. In contrast, treatment with the PTD-XIAP-soaked gel reduced infarct volumes by 51% (10.98±1.48 mm³). The protection was efficient at almost all rostro-caudal levels of the lesion, with strongest effects at the anterior pole (FIG. 3A, right panel). Despite the absence of reperfusion, a 26% reduction of lesion volumes (14.95±1.48 mm³ versus 20.35±1.48 mm³) was still observed at 7 days, with a protection restricted to levels located below the gel (FIG. 3B). FIG. 3C shows examples of TTC staining. When injected 30 min before arterial occlusion, PTD-BIR2 and PTD-BIR3/RING reduced infarction by 29% (15.33±1.31 mm3) and (45%; 11.83±1.33 mm3; FIG. 3C, left panel), respectively. Maximal protection was again observed at anterior levels for both proteins (FIG. 3C, right panel). PTD-BIR3/RING was still efficient when administered 30 min (35%, 14.04±1.75 mm3) or 3 h (17%, 17.98±1.41 mm3) after dMCAO (FIG. 3D). Examples of TTC staining used for measuring infarct volumes are shown in FIG. 3E for PTD-XIAP.

Analysis of the effects of increasing concentrations of PTD-BIR3/RING injected 30 min post-dMCAO revealed a linear (p=0.013) correlation with the protection of infarct volumes (FIG. 9).

PTD-XIAP Reduces Functional Deficits Induced by dMCAO

On the Morris water maze test, no main effect of treatment, by PTD-XIAP or time, and no treatment by time interaction was detected in the early phase (FIG. 4A, left). Acquisition of the spatial strategy was equivalent in all groups, as shown by the two probe trials (FIG. 4C; F_{(3,72)}=40.61, p<0.001). In contrast, swim speed was significantly increased at the end of the early phase in dMCAO animals (FIG. 4B, p<0.01), and was normalized by PTD-XIAP treatment (F_{(2,144)}=4.79, p<0.01).

In the late phase, a main effect of time (F_{(6,126)}=5.07, p<0.0001), and treatment (F_{(2,126)}=5.19, p<0.007) was seen on the path-length, that was normalized by the protein (p<0.03). Swim speed was kept elevated in dMCAO animals, and the effect of treatment on this parameter was progressively lost (FIG. 4B). Treatment by PTD-XIAP also normalized the number of successful trials to find the platform in less than 8 m (FIG. 4A, inserts; p<0.01).

### Effects of PTD-XIAPs Involve Caspase Inhibition and Activation of Transcription Factors

Distal MCAO induced a biphasic activation of caspase-3 with peaks at 1 h and 6 h (FIG. 5A) and a progressive activation of caspase-9 during the first 3 h (FIG. 5B). All proteins significantly reduced the activation of both caspases (FIGS. 5A-5B). In agreement with the low levels of PTD-XIAP in brain structures, caspase-3 activity was not significantly decreased at 1 h. Results of activity assays were confirmed by immunoblotting against caspase active forms (FIGS. 5A-B).

Next, we examined the DNA binding activities of NF-κB and API by EMSA (FIG. 6). NF-κB DNA binding transiently increased after dMCAO (FIGS. 6A-6C) and was further increased by PTD-XIAP treatment. Increased NF-κB DNA binding was also detected in the contralateral cortex of PTD-XIAP-treated mice. No binding activity was observed in cytosolic extracts. Addition of a 100-fold excess of unlabeled NF-κB-binding oligonucleotides abolished DNA-protein complex, and no changes were observed after incubation with unrelated oligonucleotides (TFIID). No binding was observed in the absence of proteins, which confirmed the specificity of the binding in our experimental conditions. Pre-incubation of nuclear extracts from PTD-XIAP-treated mice with anti-NF-κB antibodies strongly reduced NF-κB DNA-binding.

No or low levels of AP1 DNA-binding activity were detected in control or PTD-XIAP-treated mice 1 h after dMCAO, nor in contralateral sides or in cytosolic fractions of PTD-GFP- and PTD-XIAP-treated mice (FIGS. 6D-6E). A significant shift was observed 6 h post-pMCAO in PTD-XIAP-treated mice (FIG. 6D). In competition experiments, only the unlabeled AP1 binding consensus sequence reduced AP1 binding activity, whereas the unrelated OCT-1-binding oligonucleotides did not (FIG. 6E). A “supershift”, dose-dependent, band was observed by addition of c-Fos antibodies (2 or 4 μg) to nuclear extracts, confirming the specificity of the assay (FIG. 6F).

### Example 2

### PTD-BIR3/RING Fusion Proteins Against Transient Cerebral Ischemia Neuroprotective Effect of PTD-BIR3/RING in Cerebral Ischemia with Reperfusion

Neuroprotective activity of the PTD-BIR3/RING fusion protein was evaluated in a structural and functional way in a model of sylvian infarct with reperfusion in rat. Occlusion of the MCA is performed by intraluminal insertion of a nylon monofilament into the internal carotid artery up to the branching off point of the MCA (Hata et al., 1998). The filament is withdrawn after 2 h of occlusion which lets the recirculation of blood flow in the ischemic territory. Occlusion is monitored by laser Doppler flowmetry. This technique differs from the occlusion technique by electro-coagulation used in mouse because it results in a lesion spread to the striatum structures that order motility, additional to the cortical lesion. Two modes of treatment have been analyzed: administering before occlusion, which means in preventive conditions and without a rupture of the brain-blood barrier (BBB), and administering during reperfusion, in order to mime the conditions of an administering coupled to a thrombolytic drug in human. In both cases, the protein was injected by intravenous delivery. Motor performances were analyzed by an apomorphine-induced rotation test (rotation behaviour after injection of 0.5 mg/kg of apomorphine). Sensori-motor performances were evaluated by the adhesive removal test (ART). Animals treated before the arterial occlusion showed a significant decrease in the lesion volume induced after 2 hours of occlusion, as well as a protection from oedema (FIG. 8A). Animals treated during the reperfusion showed the same decrease in the infarct volume associated with a significant recovery of the behaviour deficits induced by ischemia (FIG. 8B-D).

a) Administration 30 Minutes Before the Arterial Occlusion.

The injection of the PTD-BIR3/RING protein decreases of 20% the volume of the cortical lesion analyzed after 24 hours and of 18% the volume of the striatum lesion. The protein decreases of 24% the total oedema, which reflects the volume of tissue running a risk at long term. This confirms the results obtained in mouse with an injection of PTD-BIR3/RING before the permanent occlusion of the MCA and it also confirms the fact that the fusion protein is able to cross efficiently the BBB and the cellular membranes when it is injected by the systemic way.

b) Administration During Reperfusion.

The protection from a unique injection of 40(160) μg of PTD-BIR3/RING in the internal carotid during reperfusion is of 25(31%) %. The later is correlated to a rectification of close to 100% of the rotation behaviour induced by an injection of 0.5 mg/kg of apomorphine (FIG. 8C). Disruption of sensori-motor functions are also protected by PTD-BIR3/RING (FIG. 8D). Reduction of infarct volumes were major in cortical areas (72%) with less efficacy on striatal tissues (28%). Reduction of functional deficits was dramatic after one month, indicating long-lasting, probably permanent, efficacy of PTD-BIR3/RING.

### Analysis of the Neuroprotective Effect of PTD-BIR3/RING on Hypoxia-Induced Neuronal Death in Hippocampal Brain Slices

The in vitro slice model was used to analyze the potential of PTD-BIR3/RING to limit hypoxia-induced neuronal death. Organotypic slice cultures were produced from rat according to Noraberg et al, (Noraberg et al., 2005). Exposition to 3 hours hypoxia resulted in total degeneration of the CA1 hippocampal area within 24 h. Dead cells were visualized by incubation of slices with propidium iodide (PI) fluorescence. Living cells are not permeable to PI, which penetrates into dead cells with compromised membrane integrity. Fluorescence was quantified using the “Image 1.55” analysis software (National Institutes of Health). No PI incorporation is observed before induction of hypoxia in control (FIG. 16A) or PTD-BIR3/RING-treated (FIG. 16B) sections. Dramatic cell death is observed on hippocampal sections 24 h following hypoxia (FIG. 16C). Treatment with PTD-BIR3/RING results in a significant reduction of PI-labeled cells (FIG. 16D). The quantification of the protective effects of PTD-BIR3/RING (FIG. 16E) and the dose-response curve (FIG. 16F) shows that maximal protection is observed for a 5 ng/μL dose.

### Intracellular Signaling of PTD-BIR3/RING: Inhibition of the Endoplasmic Reticulum-Related Death Pathway

In addition to death pathways linked to the death receptors of the TNFR family and to the mitochondrial pathway, the endoplasmic reticulum (ER) stress pathway plays a major role in ischemic cell death (Qi et al., 2004). The ER is an important storage site of calcium. Ischemia induces massive increase of intracellular calcium that leads to the activation of several ER stress-related mechanisms, among which is the activation of caspase-12 and the unfolded protein response (UPR). UPR involves a number of ER stress sensors that include the activating transcription factor (ATF)-6. ATF-6 up-regulates the transcription of glucose regulated proteins GRP78 and GRP94 genes (Yu et al., 1999; De Gracia et al., 2002).

No changes of pro-caspase-12, GRP78 and GRP94 levels were observed 1 h and 3 h after MCAO. Six hours after MCA occlusion, levels of pro-caspase-12 were decreased in MCAO mice compared to non-operated mice, and active caspase-12 was detected (FIGS. 17A, 17B and 17C). High levels were still observed at 24 h. Administration of PTD-BIR3/RING inhibited ischemia-induced caspase-12 processing into the active form. The active GRP94 protein (80 kDa fragment) was detected in PTD-GFP-treated mice and was absent in PTD-BIR3/RING-treated mice.

### Absence of Effect of PTD-BIR3/RING on Cell Proliferation In Vitro

Adverse effects of apoptosis inhibition include uncontrolled proliferation of tumoral cells. The possibility of inducing unwanted proliferation was analyzed on the highly proliferative ND7 cell line. PTD-BIR3/RING was applied on 0.5×10³ and 5×10³ cells for 24 (0.5×10³) or 36 (5×10³) hours. The FIGS. 19A and 19B show that treatment with PTD-BIR3/RING had no impact on the proliferative pattern after 1 and 2 days (FIG. 18A) or 1 and 3 days (FIG. 18B) for low- or high-density cultures, respectively.

### Protective Effects of PTD-BIR3/RING In Vitro on Neurospheres

The neuroprotective effects of PTD-BIR3/RING have been evaluated in vitro on neurosphere subjected to etoposide-induced cell death (FIG. 10). A clear neuroprotective effect is observed on apoptotic cells identified with fluorescence-activated cell sorting (FACS) by Annexin V (Annexin V-FITC kit, Beckman Coulter) labeling and absence of membrane breakdown indicated by lack of propidium iodide incorporation.

### Lack of Adverse Effects on the Lymphocytes

The occurrence of adverse repercussions of the treatment by PTD-BIR3/RING (single injection of 0.8 μg/g) in vivo was analysed in mice subjected to dMCAO. Lymphocytic populations were analysed in the spleen, the thymus and lymph nodes by FACS. No modification of cell distribution and of lymphocytes maturation was observed (FIG. 11). No increased animal mortality was observed in the treated group.

### Example 3

### Cardioprotective Effect of PTD-BIR3/RING

The PTD-BIR3/RING protein was tested in a murine model of myocardial infarction with spectacular results. PTD-BIR3/RING presents a protective capacity higher than the maximal protection never described in this model, which is obtained by a preconditioning. Cardiac ischemia is performed in mouse by 30 min of arterial occlusion, followed by 24 to 72 h reperfusion. The fusion protein was administered intravenously 30 min or 3 h after reperfusion.

The PTD-BIR3/RING protein was tested in the following conditions:

Experimental Model

Ischemia is induced by insertion in the myocardium of an occlusive thread, which encircles tightly the coronary artery (CAO: Coronary Artery Occlusion), in C57BI/6 male mice of 8 weeks (25-30 g), under anaesthesia. Occlusion was induced by tightening of the thread for 30 min. Loosening of the thread allows reperfusion of the myocardium. Intracardiac perfusion of Evans blue reveals three regions: a blue region that corresponds to the region where the blood flow circulates normally (healthy tissue), a pinkish region, where the tissue is still viable but suffering hypoxia (area at risk, AR), and a white region where the tissue is dead (Infarct area, IA). The area at risk is quantified as a percentage of the total volume of the left ventricle (IA % LV). The volume of degenerated tissue is measured as a percentage of the area at risk (AI % AR).

Treatment

Mice receive a single injection of PBS or of PTD-BIR3/RING (0.8 mg/kg) into the carotid 30 min or 3 h after reperfusion. The animals are sacrificed and the IA and AR are measured for each animal.

Results

FIG. 7A: The average volume of the area at risk is the same in the three experimental groups: White: normal mice; thin hatches: mice after ischemia and treated with PTD-BIR3/RING at 30 min; large hatches: mice after ischemia and treated with PTD-BIR3/RING at 3 h.

FIG. 7B: Treatment with PTD-BIR3/RING 3 min after the beginning of the reperfusion significantly decreases (26 versus 43 mm³) the volume of infarct tissue by comparison with the volume of the area at risk. An important protection is still observed when the protein is administered 3 h after reperfusion.

FIG. 7C: Treatment with PTD-BIR3/RING significantly reduces of the ratio IA/volume of the left ventricle.

FIG. 7D: Administration of PTD-BIR3/RING inhibits ischemia-induced activation of caspase-3, -8, and -9. Caspase-9 is a known target of XIAP's BIR3 domain. Inhibition of caspase-9 after administration of PTD-BIR3/RING (FIG. 16) was an expected finding. Inhibition of caspase-9 activity indicates that the protein has kept its biological activity after entering cardiac tissues.

Inhibition of caspase-3 is the role of the BIR2 domain in the XIAP molecule, but has been reported when excess amounts of BIR3 are incubated in presence of caspases-3 in vitro. The Inventors also observed an inhibition of caspase-3 in ischemic brain tissues treated with PTD-BIR3/RING. Inhibition of caspase-3 by PTD-BIR3/RING (FIG. 16) is an important finding, since caspase-3 is the main executor caspase in the apoptotic process and plays a key role in ischemic cell death.

Caspase-8 is activated by the so-called “death receptors” that belong to the family of the tumor necrosis factor receptor-1 (TNF-R1). Activation of TNFR-1 induces activation of the pro-caspase-8. Active caspase-8 has a number of targets, including caspase-3, the nuclear PARP-1 and -2, and Bid. Inhibition of caspase-8 by XIAP or any of its domains (FIG. 16) is a new finding. It shows that PTD-BIR3/RING has a wide range of actions on caspases, and can interfere with both the extrinsic (death-receptors-mediated) and intrinsic (mitochondrial) pathways.

Reduction of Apotosis in Cardiomyocytes after Administration of PTD-BIR3/RING in Cardiac Ischemia

TUNEL labeling of myocardium after 30 min occlusion of the coronary artery in mice reveals apoptotic cells (FIGS. 12 A and 12 B, condition 1). The apoptotic cells appear in brown. Treatment with PTD-BIR3/RING significantly reduces the number of TUNEL-positive cardiomyocytes (FIGS. 12 A and 12 B, condition 2).

### Administration of PTD-BIR3/RING Increases the Ratio of Phosphorylated Versus Non-Phosphorylated Forms of the Serin-Threonin Kinase Akt, and the Pro-Apoptotic Bad.

Akt/PKB, a P13-kinase activated protein kinase, is a principal mediator of cell survival. Phosphorylated Akt plays a major role in cell protection by phosphorylation of a number of targets. Akt also affects the transcriptional response to apoptotic stimuli, for example, by affecting on Forkhead factors and the activity of the p53 family. In addition, novel connections between the metabolic effects of Akt/PKB and its control of survival have recently been made (Song et al., 2005).

Among phosphorylated-Akt targets is the pro-apoptotic member of the Bcl-2 family, Bad. Bad is kept inactive in the cytoplasm in unphosphorylated form. During apoptosis, Bad is phosphorylated to participate in the activation of the apoptotic mitochondrial pathway. Active (unphosphorylated) Bad induces apoptosis by binding to and inhibiting antiapoptotic Bcl-2 family members, such as BC1-XL, thereby allowing two other proapoptotic members, Bak and Bax, to aggregate. This induces the release of cytochrome c, caspase activation, and apoptosis. Phosphorylation of endogenous Bad by survival kinases is necessary to raise the threshold at which mitochondria release cytochrome c in response to apoptotic stimuli. In this way, Bad phosphorylation serves as a sensor for survival signaling and determines the level of apoptotic input a cell has to be exposed to in order to undergo apoptosis.

The state of Akt phosphorylation was analyzed during cardiac ischemia (FIG. 13A, 15). Administration of PTD-BIR3/RING 30 min after reperfusion following a 30 min CAO significantly increased the phosphorylated form of Akt. This increase is correlated with a significant increase of the phosphorylated form of Bad (FIG. 13B).

### Administration of PTD-BIR3/RING Decreases Bax and the Truncated Form of Bid.

Bid and Bax are pro-apoptotic members of the Bcl2 family. Bid is targeted for proteolytic cleavage by caspase-8. The resulting truncated form, or Bidt, is highly active and participates in the translocation of Bax into the outer mitochondrial membrane, leading to pore formation and leakage of apoptosis activators, like cytochrome c (Korsmeyer et al., 2000). The reduction of Bidt and bax concentrations in the cytoplasm indicates a protective effect of PTD-BIR3/RING through regulation of the mitochondrial pathway. FIGS. 14A and 14B show a reduction of Bidt and Bax in the ischemic cardiac muscle after administration of PTD-BIR3/RING 30 min after CAR, showing inhibition of the mitochondrial apoptotic pathway.

Quantification of the Modifications of Akt, Phosphorylated Akt (P-Akt), Truncated Bid (Bidt) and Bax after 30 min of CAO.

PTD-BIR3/RING (0.8 mg/kg in 100 μl) or PBS (100 μl) was administered intravenously 30 m in after reperfusion. Quantification of Western blot data confirms the reduction of Bidt and Bax contents in PTD-BIR3/RING-treated myocardium, and the increase in phosphorylated Akt.

The effects of XIAP on Akt and Bad phosphorylation, and on the caspase-8/Bid/Bax signalization pathway have been identified for the first time in this study.

These experiments confirm the cytoprotective potential of the PTD-BIR3/RING protein, which is significant on cardiomyocytes as well as on neuronal cells. These results confirm the use of the fusion proteins according to the present invention for the treatment of cardiac ischemia following an arterial occlusion or a heart failure, and against degenerative lesions of the cerebral and cardiac tissues in general. No life-threatening of severe adverse effects have been noted so far.

According to the results of the Inventors, PTD-BIR3/RING exerts its anti-ischemic effect through activation of survival pathways correlated with inhibition of pro-apoptotic pathways. Survival pathways include activation of Akt/PKB, activation of transcription factors (NFκB and AP1), and decrease of Bad contents. Inhibition of pro-apoptotic pathways includes caspase inhibition, phosphorylation of Bad, and reduction of the truncated form of Bid. Importantly, caspase inhibition is not restricted to the well known effects on caspase-3 and -9, but involves inhibition of caspase-12, leading to reduction of endoplasmic reticulum stress-related mechanisms, and involves inhibition of caspase-8, leading to reduction of the <<death receptor pathway>> of apoptosis.

### CONCLUSION

The results of the Inventors confirm the potential of the PTD fusion proteins strategy. PTD-XIAP concentrated at lesion sites protected underlying cortical regions up to one week, and shorter constructs injected intravenously significantly protected cerebral degeneration, showing that the strategy is efficient even in the absence of reperfusion.

The nuclear localization of endogenous XIAP, reported in two studies (Liston et al., 2001; Wang et al., 2004), was not observed in this work, and endogenous XIAP accumulated around the nucleus.

Occlusion in this model is performed distally on the cortical branch of the MCA. This procedure spares striatal structures and induces no discrete motor or sensori-motor deficits (Jadecola et al., 1997). It must be mentioned that models referred to as “permanent MCAO” generally involve more proximal occlusions with blood flow arrest in both cortical and striatal MCA branches, leading to conspicuous motor deficits. In our model, the two probe trials correlated previous observations by confirming that dMCAO animals have a correct spatial learning. However, long-term follow-up of ischemic animals revealed consistent modifications of two test parameters, increased swim speed, and inability to reduce path length to the platform below the 8 meters threshold. Coupled to a correct spatial strategy, increased distance to the target reveals a lack of precision that can be attributed to attention deficits. According to the lesion location in this model, attention deficits are correlated to loss of temporo-parietal regions in humans (Chambers et al., 2004). Treatment with PTD-XIAP significantly improved this score and reduced the distance to the platform, indicating recovery of functional deficits induced by dMCAO.

Activation of NF-κB in the present model was transient, which is a prerequisite for promoting cell survival, when long-lasting NF-κB activity has been associated with cell death (Clemens, 2000). Since XIAP is among NF-κB gene targets (Stehlik et al., 1998; Zou et al., 2004), its activation may also result in increased production of endogenous XIAP, adding to the neuroprotective efficacy of the exogenous PTD-XIAP.

The Inventors show that administration of XIAP can induces AP1 activation in vivo in a neuroprotective context. API activity is stimulated by phosphorylation of the c-Jun transactivation domain by JNK (Yang et al., 1997), which is selectively activated by XIAP (Sanna et al., 1998). In an ischemic context, XIAP might lead, through JNK activation, to increased AP1 DNA binding and transcription of genes with neuroprotective properties, among which are late effector genes such as nerve growth factor (Guégan et al., 1998), brain-derived neurotrophic factor (Dechant and Neumann, 2002), or basic fibroblast growth factor (Lindvall et al., 1992).

Results obtained with the PTD-XIAP fusion protein confirm that protein therapy using molecules acting on synergistic neuroprotective pathways is a promising alternative to monomodal approaches in limiting brain lesions with complex aetiology such as stroke.

### REFERENCES

Benchoua, A., Guegan, C., Couriaud, C., Hosseini, H., Sampaio, N., Morin, D., Onteniente, B., 2001. Specific caspase pathways are activated in the two stages of cerebral infarction. J. Neurosci. 21, 7127-7134.
Clemens, J. A., 2000. Cerebral ischemia: gene activation, neuronal injury, and the protective role of antioxidants? Free Rad. Biol. Med. 28, 1526-1531.
Chambers, C. D., Stokes, M. G., Mattingley, J. B., 2004. Modality-specific control of strategic spatial attention in parietal cortex. Neuron. 44, 925-930.
Dechant, G., Neumann, H., 2002. Neurotrophins. Review. Adv. Exp. Med. Biol. 513, 303-334.
Datta et al. 2002. Survival factor-mediated Bad phosphorylation raises the mitochondrial threshold for apoptosis. Review. Dev. Cell. November; 3(5):631-643.
DeGracia D J, Kumar R, Owen C R, Krause G S, White BC. Molecular pathways of protein synthesis inhibition during brain reperfusion: implications for neuronal survival or death. J Cereb Blood Flow Metab. 2002 February; 22(2):127-41. Review.
Deveraux, Q. L., Leo, E., Stennicke, H. R., Welsh, K., Salvesen, G. S., Reed, J. C., 1999. Cleavage of human inhibitor of apoptosis protein XIAP results in fragments with distinct specificities for caspases. EMBO J. 18, 5242-5251.
Dietz, G. P. H., Kilic, E., Bähr, M., 2002. Inhibition of neuronal apoptosis in vitro and in vivo using TAT-mediated protein transduction. Mol. Cell. Neurosci. 21, 29-37.
Friedlander, R. M., 2003. Apoptosis and caspases in neurodegenerative diseases. N. Engl. J. Med. 48, 1365-1375.
Gotti, B., Benavides, J., Mackenzie, E. T., Scatton, B., 1990. The pharmacotherapy of focal cortical ischaemia in the mouse. Brain Res. 522, 290-307.
Guégan, C., Onténiente, B., Makiura, Y., Merad-Boudia, M., Ceballos-Picot, I., Sola, B., 1998. Reduction of cortical infarction and impairment of apoptosis in NGF-transgenic mice subjected to permanent focal ischemia. Mol. Brain. Res. 55, 133-140.
Hata R, Mies G, Wiessner C, Fritze K, Hesselbarth D, Brinker G, Hossmann K A. A reproducible model of middle cerebral artery occlusion in mice: hemodynamic, biochemical, and magnetic resonance imaging. J Cereb Blood Flow Metab. 1998 April; 18(4):367-75.
Holcik, M., Gibson, M., Korneluk, R. G., 2001. XIAP: Apoptotic brake and promising therapeutic target. Apoptosis 6, 253-261.
Huang, H., Joazeiro, C. A., Bonfoco, E., Kamada, S., Leverson, J. D., Hunter, T., 2000. The inhibitor of apoptosis, cIAP2, functions as a ubiquitin-protein ligase and promotes in vitro monoubiquitination of caspases 3 and 7. J. Biol. Chem. 275, 26661-2664.
Iadecola, C., Zhang, F., Casey, R., Nagayama, M., Ross, M. E., 1997. Delayed reduction of ischemic brain injury and neurological deficits in mice lacking the inducible nitric oxide synthase gene. J. Neurosci. 17, 9157-9164.
Korsmeyer S J, Wei M C, Saito M, Weiler S, Oh K J, Schlesinger P H. 2000. Pro-apoptotic cascade activates BID, which oligomerizes BAK or BAX into pores that result in the release of cytochrome c. Review Cell Death Differ. December; 7(12):1166-73.
Lewis, J., Burstein, E., Refey, S. B., Brattoni, S. B., Roberts, A. B., Duckett, C. S., 2004. Uncoupling the signaling and caspase-inhibitory properties of the X chromosome-linked inhibitor of apoptosis. J. Biol. Chem. 279, 9023-9029.
Lindvall, O., Ernfors, P., Bengzon, J., Kokaia, Z., Smith, M. L., Siesjo, B. K., Persson, H., 1992. Differential regulation of mRNAs for nerve growth factor, brain-derived neurotrophic factor, and neurotrophin 3 in the adult rat brain following cerebral ischemia and hypoglycemic coma. Proc. Natl. Acad. Sci. USA. 89, 648-652.
Liston, P., Fong, W. G., Kelly, N. L., Toji, S., Miyazaki, T., Conte, D., Tamai, K., Craig, C. G., McBurney, M. W., Komeluk, R. G., 2001. Identification of XAF1 as an antagonist of XIAP anti-Caspase activity. Nat. Cell Biol. 3, 128-133.
Nakane, P. K., 1968 Simultaneous localization of multiple tissue antigens using the peroxidase-labeled antibody method: a study on pituitary glands of the rat. J. Histochem. Cytochem. 16, 557-560.
Noraberg J, Poulsen F R, Blaabjerg M, Kristensen B W, Bonde C, Montero M, Meyer M, Gramsbergen J B, Zimmer J. Organotypic hippocampal slice cultures for studies of brain damage, neuroprotection and neurorepair. Curr Drug Targets CNS Neurol Disord. 2005 August; 4(4):435-52. Review.
Onténiente, B., 2004. Natural and synthetic inhibitors of caspases: targets for novel drugs. Curr. Drug Targets CNS Neurol. Disord. 3, 333-340.
Plesnila, N., Moskowitz, M. A., 2000. Caspases in cerebral ischemia. In: Pharmacology of cerebral ischemia (Krieglstein J, Klumpp S, eds), pp 137-133, Stuttgart: Medpharm Scientific.
Qi X, Okuma Y, Hosoi T, Nomura Y. Edaravone protects against hypoxia/ischemia-induced endoplasmic reticulum dysfunction. J Pharmacol Exp Ther. 2004 October; 311(1):388-93.
Reynolds B. A., Weiss S. (1992) Generation of neurons and astrocytes from isolated cells of the adult mammalian central nervous system. Science 255:1707-10.
Reed J C. 2006. Proapoptotic multidomain Bcl-2/Bax-family proteins: mechanisms, physiological roles, and therapeutic opportunities. Review Cell Death Differ. Aug; 13(8):1378-86. Epub 2006 Jun. 2.
Sanna, M. G., Duckett, C. S., Richter, B. W., Thompson, C. B., Ulevitch, R. J., 1998. Selective activation of JNK1 is necessary for the anti-apoptotic activity of hILP. Proc. Natl. Acad. Sci. USA. 95, 6015-6020.
Schwarze, S., Ho, A., Vocero-Akbani, A., Dowdy, S., 1999. In vivo protein transduction: delivery of a biologically active protein into the mouse. Science 285, 1569-1572.
Stehlik, C., de Martin, R., Kumabashiri, T., Schmid, J. A., Binder, B. R., Lipp, J., 1998 Nuclear factor (NF)-kappaB-regulated X-chromosome-linked iap gene expression protectors endothelial cells from tumor necrosis factor alpha-induced apoptosis. J. Exp. Med. 188, 211-216.
Song G., Ouyang G., Bao S., 2005. The activation of Akt/PKB signaling pathway and cell survival. J. Cell. Mol. Med. Vol 9, No 1, 2005 pp. 59-71
Trapp, T., Korhonen, L., Besselmann, M., Martinez, R., Mercer, E. A., Lindholm, D., 2003. Transgenic mice overexpressing XIAP in neurons show better outcome after transient cerebral ischemia. Mol. Cell. Neurosci. 23, 302-313.
Vaux, D. L., Silke, J., 2003. Mammalian mitochondrial IAP binding proteins. Biochem. Biophys. Res. Comm. 304, 499-504.
Wang, X., Zhu, C., Wang, X., Hagberg, H., Korhonen, L., Sandberg, M., Lindholm, D., Blomgren, K., 2004. X-linked inhibitor of apoptosis (XIAP) protein protects against caspase activation and tissue loss after neonatal hypoxia-ischemia. Neurobiol. Dis. 16, 179-189.
Xu, D., Bureau, Y., McIntyre, D. C., Nicholson, D. W., Liston, P., Zhu, Y., Fong, W. G., Crocker, S. J., Korneluk, R. G., Robertson, G. S., 1999. Attenuation of ischemia-induced cellular and behavioral deficits by X chromosome-linked inhibitor of apoptosis protein overexpression in the rat hippocampus. J. Neurosci. 19, 5026-5033.
Yang, D., Tournier, C., Wysk, M., Lu, H. T., Xu, J., Davis, R. J., Flavell, R. A., 1997. Targeted disruption of the MKK4 gene causes embryonic death, inhibition of c-Jun NH₂-terminal kinase activation, and defects in AP-1 transcriptional activity. Proc. Natl. Acad. Sci. USA. 94, 3004-3009.
Yang, Y., Fang, S., Jensen, J. P., Weissman, A. M., Ashwell, J. D., 2000. Ubiquitin protein ligase activity of IAPS and their degradation in proteasomes in response to apoptotic stimuli. Science 288, 874-877.
Yu Z, Luo H, Fu W, Mattson M P. The endoplasmic reticulum stress-responsive protein GRP78 protects neurons against excitotoxicity and apoptosis: suppression of oxidative stress and stabilization of calcium homeostasis. Exp Neurol. 1999 February; 155(2):302-14.
Zou, T., Rao, J. N., Guo, X., Liu, L., Zhang, H. M., Strauch, E. D., Bass, B. L., Wang, J. Y., 2004. NF-kappaB-mediated IAP expression induces resistance of intestinal epithelial cells to apoptosis after polyamine depletion. Am. J. Physiol. Cell. Physiol. 286, C1009-1018.

1-22. (canceled)

23. A method for the prevention or the treatment of pathologies resulting from ischemia by using an element chosen among the group consisting of:
a fusion protein comprising or constituted of
at least one peptide sequence having membrane transducing properties and,
at least one peptide sequence having cell survival properties;
a homologous protein derived from said fusion protein by insertion, deletion, or substitution of at least one amino acid, provided that said homologous protein presents an identity percentage of at least 85%, and preferably of at least 89%, with respect to said fusion protein and presents membrane transducing and cell survival properties, and
a nucleic acid coding for said fusion protein or for said homologous protein.

24. A method according to claim 23, wherein the peptide sequence having membrane transducing properties is the protein transduction domain (PTD) of the HIV TAT protein, such as the protein transduction domain (PTD) of the HIV TAT protein represented by SEQ ID NO: 2.

25. A method according to claim 24, for the prevention or the treatment of pathologies resulting from myocardial ischemia.

26. A method according to claim 25, wherein the peptide sequence having cell survival properties is selected from the list comprising:
the X chromosome-linked apoptosis inhibitor protein (XIAP) or fragments thereof having cell survival properties, including the BIR2, BIR3-RING, or BIR3-RING linker domains of XIAP, and
the Flice inhibitory protein (FLIP) or fragments thereof having cell survival properties.

27. A method according to claim 26, wherein the peptide sequence having cell survival properties is selected from the list comprising:
the XIAP represented by SEQ ID NO: 4 (rat),
the XIAP represented by SEQ ID NO: 6 (human),
the BIR2 domain represented by SEQ ID NO: 8 (rat),
the BIR2 domain represented by SEQ ID NO: 44 (human),
the BIR3-RING domain represented by SEQ ID NO: 10 (rat),
the BIR3-RING domain represented by SEQ ID NO: 12 (human),
the linker domain between the BIR3 and RING domains represented by SEQ ID NO: 36 (rat),
the linker domain between the BIR3 and RING domains represented by SEQ ID NO: 38 (human),
the FLIP represented by SEQ ID NO: 14 (FlipL) (mouse),
the FLIP represented by SEQ ID NO: 16 (FlipS) (mouse), and
the FLIP represented by SEQ ID NO: 18 (Flip control) (mouse).

28. A method according to claim 27, wherein the fusion protein is selected from the list comprising:
PTD-XIAP represented by SEQ ID NO: 20, corresponding to the fusion of SEQ ID NO: 2 and SEQ ID NO: 4,
PTD-XIAP represented by SEQ ID NO: 22, corresponding to the fusion of SEQ ID NO: 2 and SEQ ID NO: 6,
PTD-BIR2 represented by SEQ ID NO: 24, corresponding to the fusion of SEQ ID NO: 2 and SEQ ID NO: 8,
PTD-BIR2 represented by SEQ ID NO: 46, corresponding to the fusion of SEQ ID NO: 2 and SEQ ID NO: 44,
PTD-BIR3-RING represented by SEQ ID NO: 26, corresponding to the fusion of SEQ ID NO: 2 and SEQ ID NO: 10,
PTD-BIR3-RING represented by SEQ ID NO: 28, corresponding to the fusion of SEQ ID NO: 2 and SEQ ID NO: 12,
PTD-(BIR3-RING linker) represented by SEQ ID NO: 40, corresponding to the fusion of SEQ ID NO: 2 and SEQ ID NO: 36,
PTD-(BIR3-RING linker) represented by SEQ ID NO: 42, corresponding to the fusion of SEQ ID NO: 2 and SEQ ID NO: 38,
PTD-FLIP represented by SEQ ID NO: 30, corresponding to the fusion of SEQ ID NO: 2 and SEQ ID NO: 14,
PTD-FLIP represented by SEQ ID NO: 32, corresponding to the fusion of SEQ ID NO: 2 and SEQ ID NO: 16, and
PTD-FLIP represented by SEQ ID NO: 34, corresponding to the fusion of SEQ ID NO: 2 and SEQ ID NO: 18.

29. A method according to claim 28, wherein the peptide sequence having cell survival properties comprises at least one XIAP fragment having cell survival chosen among:
the BIR2 domain, such as the BIR2 domain represented by SEQ ID NO: 8,
the BIR3-RING domain, including:
the BIR3-RING domain represented by SEQ ID NO: 10, and
the BIR3-RING domain represented by SEQ ID NO: 12, and,
the BIR3-RING linker domain, including:
the BIR3-RING linker domain represented by SEQ ID NO:36, and
the BIR3-RING linker domain represented by SEQ ID NO:38.

30. A method according to claim 29, wherein the fusion protein is:
PTD-BIR2 represented by SEQ ID NO: 24,
PTD-BIR2 represented by SEQ ID NO: 46,
PTD-BIR3-RING represented by SEQ ID NO: 26,
PTD-BIR3-RING represented by SEQ ID NO: 28,
PTD-(BIR3-RING linker) represented by SEQ ID NO: 40, and
PTD-(BIR3-RING linker) represented by SEQ ID NO: 42.

31. A method according to claim 24, for the prevention or the treatment of pathologies resulting from cerebral ischemia.

32. A method according to claim 31, wherein the peptide sequence having cell survival properties is selected from the list comprising:
human X chromosome-linked apoptosis inhibitor protein (XIAP) having cell survival properties,
fragments of the X chromosome-linked apoptosis inhibitor protein (XIAP) having cell survival properties, such as the BIR2, or BIR3-RING, or BIR3-RING linker domains of XIAP, and
fragments of the Flice inhibitory protein (FLIP) having cell survival properties.

33. A method according claim 32, wherein the peptide sequence having cell survival properties comprises at least one XIAP fragment having cell survival chosen among:
the BIR2 domain, such as the BIR2 domain represented by SEQ ID NO: 8,
the BIR3-RING domain, including
the BIR3-RING domain represented by SEQ ID NO: 10, and
the BIR3-RING domain represented by SEQ ID NO: 12, and,
the BIR3-RING linker domain, including
the BIR3-RING linker domain represented by SEQ ID NO:36, and
the BIR3-RING linker domain represented by SEQ ID NO:38.

34. A method according to claim 33, wherein the fusion protein is:
PTD-BIR2 represented by SEQ ID NO: 24,
PTD-BIR3-RING represented by SEQ ID NO: 26,
PTD-BIR3-RING represented by SEQ ID NO: 28,
PTD-(BIR3-RING linker) represented by SEQ ID NO: 40, and
PTD-(BIR3-RING linker) represented by SEQ ID NO: 42.

35. A pharmaceutical composition comprising an active substance chosen among the group consisting of:
a fusion protein comprising or constituted of
at least one peptide sequence having membrane transducing properties, and
at least one peptide sequence having cell survival properties; and,
a homologous protein derived from said fusion protein by insertion, deletion, or substitution of at least one amino acid, provided that said homologous protein presents an identity percentage of at least 85%, with respect to said fusion protein and presents membrane transducing and cell survival properties,
in association with a pharmaceutically acceptable carrier,
said pharmaceutical composition being suitable for the administration to an individual of a unit dose from 50 mg to 500 mg of the fusion protein.

36. A pharmaceutical composition according to claim 35 where the said homologous protein provides an identity percentage of at least 89% with respect to said fusion protein.

37. A pharmaceutical composition according to claim 35, wherein the peptide sequence having membrane transducing properties is the protein transduction domain (PTD) of the HIV TAT protein, such as the protein transduction domain (PTD) of the HIV TAT protein represented by SEQ ID NO: 2.

38. A pharmaceutical composition according to claim 37, wherein the peptide sequence having cell survival properties is selected from the list comprising:
the X chromosome-linked apoptosis inhibitor protein (XIAP) or fragments thereof having cell survival properties, such as the BIR2, BIR3-RING, or BIR3-RING linker domains of XIAP, and
the Flice inhibitory protein (FLIP) or fragments thereof having cell survival properties.

39. A pharmaceutical composition according to claim 38, wherein the peptide sequence having cell survival properties is selected from the list comprising:
the XIAP represented by SEQ ID NO: 4 (rat),
the XIAP represented by SEQ ID NO: 6 (human),
the BIR2 domain represented by SEQ ID NO: 8 (rat),
the BIR2 domain represented by SEQ ID NO: 44 (human),
the BIR3-RING domain represented by SEQ ID NO: 10 (rat),
the BIR3-RING domain represented by SEQ ID NO: 12 (human),
the linker domain between the BIR3 and RING domains represented by SEQ ID NO: 36 (rat),
the linker domain between the BIR3 and RING domains represented by SEQ ID NO: 38 (human),
the FLIP represented by SEQ ID NO: 14 (FlipL) (mouse),
the FLIP represented by SEQ ID NO: 16 (FlipS) (mouse), and
the FLIP represented by SEQ ID NO: 18 (Flip control) (mouse).

40. A pharmaceutical composition according to claim 39, wherein the fusion protein is selected from the list comprising:
PTD-XIAP represented by SEQ ID NO: 20, corresponding to the fusion of SEQ ID NO: 2 and SEQ ID NO: 4,
PTD-XIAP represented by SEQ ID NO: 22, corresponding to the fusion of SEQ ID NO: 2 and SEQ ID NO: 6,
PTD-BIR2 represented by SEQ ID NO: 24, corresponding to the fusion of SEQ ID NO: 2 and SEQ ID NO: 8,
PTD-BIR2 represented by SEQ ID NO: 46, corresponding to the fusion of SEQ ID NO: 2 and SEQ ID NO: 44,
PTD-BIR3-RING represented by SEQ ID NO: 26, corresponding to the fusion of SEQ ID NO: 2 and SEQ ID NO: 10,
PTD-BIR3-RING represented by SEQ ID NO: 28, corresponding to the fusion of SEQ ID NO: 2 and SEQ ID NO: 12,
PTD-(BIR3-RING linker) represented by SEQ ID NO: 40, corresponding to the fusion of SEQ ID NO: 2 and SEQ ID NO: 36,
PTD-(BIR3-RING linker) represented by SEQ ID NO: 42, corresponding to the fusion of SEQ ID NO: 2 and SEQ ID NO: 38,
PTD-FLIP represented by SEQ ID NO: 30, corresponding to the fusion of SEQ ID NO: 2 and SEQ ID NO: 14,
PTD-FLIP represented by SEQ ID NO: 32, corresponding to the fusion of SEQ ID NO: 2 and SEQ ID NO: 16, and
PTD-FLIP represented by SEQ ID NO: 34, corresponding to the fusion of SEQ ID NO: 2 and SEQ ID NO: 18.

41. A pharmaceutical composition comprising as active substance a compound chosen among the group consisting of:
a fusion protein comprising or constituted of
at least one peptide sequence having membrane transducing properties, and
at least one peptide sequence having cell survival properties selected from the list comprising:
fragments of the X chromosome-linked apoptosis inhibitor protein (XIAP) having cell survival properties, and
fragments of the Flice inhibitory protein (FLIP) having cell survival properties, and,
a homologous protein derived from said fusion protein by insertion, deletion, or substitution of at least one amino acid, provided that said homologous protein presents an identity percentage of at least 85%, with respect to said fusion protein and presents membrane transducing and cell survival properties,
in association with a pharmaceutically acceptable carrier.

42. A pharmaceutical composition according to claim 41, wherein the said homologous protein provides an identity percentage of at least 89% with respect to said fusion protein.

43. A pharmaceutical composition according to claim 41, wherein the peptide sequence having cell survival properties comprises at least one XIAP fragment having cell survival which is:
the BIR2 domain, such as the BIR2 domain represented by SEQ ID NO: 8, and
the BIR3-RING domain, including
the BIR3-RING domain represented by SEQ ID NO: 10,
the BIR3-RING domain represented by SEQ ID NO: 12,
the BIR3-RING linker domain represented by SEQ ID NO: 36 (rat),
the BIR3-RING linker domain represented by SEQ ID NO: 38 (human).

44. A pharmaceutical composition according to claim 43, wherein the fusion protein is:
PTD-BIR2 represented by SEQ ID NO: 24,
PTD-BIR2 represented by SEQ ID NO: 46,
PTD-BIR3-RING represented by SEQ ID NO: 26,
PTD-BIR3-RING represented by SEQ ID NO: 28,
PTD-(BIR3-RING linker) represented by SEQ ID NO: 40, and
PTD-(BIR3-RING linker) represented by SEQ ID NO: 42.

45. A pharmaceutical composition comprising as active substance a nucleic acid coding for a fusion protein as defined in claim 41, in association with a pharmaceutically acceptable carrier.

46. A fusion protein selected from the list comprising:
PTD-XIAP represented by SEQ ID NO: 20, corresponding to the fusion of SEQ ID NO: 2 and SEQ ID NO: 4,
PTD-XIAP represented by SEQ ID NO: 22, corresponding to the fusion of SEQ ID NO: 2 and SEQ ID NO: 6,
PTD-BIR2 represented by SEQ ID NO: 24, corresponding to the fusion of SEQ ID NO: 2 and SEQ ID NO: 8,
PTD-BIR2 represented by SEQ ID NO: 46, corresponding to the fusion of SEQ ID NO: 2 and SEQ ID NO: 44,
PTD-BIR3-RING represented by SEQ ID NO: 26, corresponding to the fusion of SEQ ID NO: 2 and SEQ ID NO: 10,
PTD-BIR3-RING represented by SEQ ID NO: 28, corresponding to the fusion of SEQ ID NO: 2 and SEQ ID NO: 12,
PTD-(BIR3-RING linker) represented by SEQ ID NO: 40, corresponding to the fusion of SEQ ID NO: 2 and SEQ ID NO: 36,
PTD-(BIR3-RING linker) represented by SEQ ID NO: 42, corresponding to the fusion of SEQ ID NO: 2 and SEQ ID NO: 38,
PTD-FLIP represented by SEQ ID NO: 30, corresponding to the fusion of SEQ ID NO: 2 and SEQ ID NO: 14,
PTD-FLIP represented by SEQ ID NO: 32, corresponding to the fusion of SEQ ID NO: 2 and SEQ ID NO: 16, and
PTD-FLIP represented by SEQ ID NO: 34, corresponding to the fusion of SEQ ID NO: 2 and SEQ ID NO: 18.


SEQUENCE LISTING (60 sequences)

SEQ ID NO: 1 - DNA, 33 bp, Homo sapiens
  CDS (1)..(33)
tacggccgcaagaaagcgcgccagcgccgccgc

SEQ ID NO: 2 - protein, 11 aa, Homo sapiens
YGRKKARQRRR

SEQ ID NO: 3 - DNA, 1491 bp, Rattus norvegicus
  CDS (1)..(1491)
atgacttttaacagttttgaaggatctagaactgttgtacctgcagacaccaataaggatgaagaatttgtagaagagtttaatagattaaaaacatttgctaacttcccaagcagcagtcctgtttcagcatcaacattggcgcgagcggggttcctctacactggtgaaggagacaccgtgcagtgtttcagttgtcacgcggcagtagatagatggcagtatggagactcagctgttggaagacacaggagaatatccccaaattgcagatttatcaatggtttttattttgaaaacggtgccacacagtctacatctcctggcatccaaaatggccagtacaaatctgaaaactgtgtgggaaacagaaatcattttgctcttgacaggccgtcggagactcatgcagattatctcctgagaactggacaggttgtagatatttcagataccatatacccgaggaacccggccatgtgtagtgaagaagccagaccgaagacgtttcagaactggccagactatgcccatttaagccccagagagttagctagtgctggactctactacacggggattgatgatcaagtgcaatgcttttgttgtggtggaaaactgaaaaattgggaaccctgtgaccgtgcctggtcagagcacaggagacactttcccaactgcttcttcgttttgggccggaatgttaatgttcgaagtgagtctggtgtgagttcagataggaatttcccaaattcaacaaattctccaagaaatccagccatggcagaatatgacgcacggatcgttacttttggaacatggctatactcagttaacaaggagcagcttgcaagagctggattttatgctttaggtgaaggtgataaagtgaagtgctttcactgtggaggagggctcacggattggaagccaagtgaagacccttgggaacagcatgctaagtggtatccagggtgtaaatatctattggatgagaagggacaagaatatataaataatattcatttaacccattcacttggggaatctgtggtaagaactgctgaaaaaacaccatcagtaactaaaaaaatcgatgataccatcttccagaatcctatggtgcaagaagctatacgaatgggattcaacttcaaggacatcaagaaaacaatggaagaaaagctccaaacatctgggagcaactatctatcacttgaggttctgattgcagatcttgtgagtgctcagaaagataattcgcaggatgagtcaagtcagacttcattgcagaaagacatcagtactgaagagcagctaaggcgcctacaagaggagaagctttgcaaaatctgtatggatagaaatattgctatagtttttgttccttgtggacatctggtcacttgtaaacagtgtgcggaagcagttgacaaatgtcccatgtgctgcacagtcattacgttcaagcaaaaaatttttatgttttaa

SEQ ID NO: 4 - protein, 496 aa, Rattus norvegicus
MTFNSFEGSRTVVPADTNKDEEFVEEFNRLKTFANFPSSSPVSASTLARAGFLYTGEGDTVQCFSCHAAVDRWQYGDSAVGRHRRISPNCRFINGFYFENGATQSTSPGIQNGQYKSENCVGNRNHFALDRPSETHADYLLRTGQVVDISDTIYPRNPAMCSEEARPKTFQNWPDYAHLSPRELASAGLYYTGIDDQVQCFCCGGKLKNWEPCDRAWSEHRRHFPNCFFVLGRNVNVRSESGVSSDRNFPNSTNSPRNPAMAEYDARIVTFGTWLYSVNKEQLARAGFYALGEGDKVKCFHCGGGLTDWKPSEDPWEQHAKWYPGCKYLLDEKGQEYINNIHLTHSLGESVVRTAEKTPSVTKKIDDTIFQNPMVQEAIRMGFNFKDIKKTMEEKLQTSGSNYLSLEVLIADLVSAQKDNSQDESSQTSLQKDISTEEQLRRLQEEKLCKICMDRNIAIVFVPCGHLVTCKQCAEAVDKCPMCCTVITFKQKIFMF

SEQ ID NO: 5 - DNA, 1622 bp, Homo sapiens
  CDS (129)..(1622)
tttccagattggggctcgggccgcgcctcctccgggaccctccccttggaccgagccgatcgccgcggggcagttcgggccggctgtcctggcgcgaaaaggtggacaagtcctattttcaagagaagatgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagatagatggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtcttaa

SEQ ID NO: 6 - protein, 497 aa, Homo sapiens
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 7 - DNA, 411 bp, Rattus norvegicus
  CDS (1)..(411)
agaaatcattttgctcttgacaggccgtcggagactcatgcagattatctcctgagaactggacaggttgtagatatttcagataccatatacccgaggaacccggccatgtgtagtgaagaagccagaccgaagacgtttcagaactggccagactatgcccatttaagccccagagagttagctagtgctggactctactacacggggattgatgatcaagtgcaatgcttttgttgtggtggaaaactgaaaaattgggaaccctgtgaccgtgcctggtcagagcacaggagacactttcccaactgcttcttcgttttgggccggaatgttaatgttcgaagtgagtctggtgtgagttcagataggaatttcccaaattcaacaaattctccaagaaatccagcc

SEQ ID NO: 8 - protein, 137 aa, Rattus norvegicus
RNHFALDRPSETHADYLLRTGQVVDISDTIYPRNPAMCSEEARPKTFQNWPDYAHLSPRELASAGLYYTGIDDQVQCFCCGGKLKNWEPCDRAWSEHRRHFPNCFFVLGRNVNVRSESGVSSDRNFPNSTNSPRNPA

SEQ ID NO: 9 - DNA, 711 bp, Rattus norvegicus
  CDS (1)..(711)
atggcagaatatgacgcacggatcgttacttttggaacatggctatactcagttaacaaggagcagcttgcaagagctggattttatgctttaggtgaaggtgataaagtgaagtgctttcactgtggaggagggctcacggattggaagccaagtgaagacccttgggaacagcatgctaagtggtatccagggtgtaaatatctattggatgagaagggacaagaatatataaataatattcatttaacccattcacttggggaatctgtggtaagaactgctgaaaaaacaccatcagtaactaaaaaaatcgatgataccatcttccagaatcctatggtgcaagaagctatacgaatgggattcaacttcaaggacatcaagaaaacaatggaagaaaagctccaaacatctgggagcaactatctatcacttgaggttctgattgcagatcttgtgagtgctcagaaagataattcgcaggatgagtcaagtcagacttcattgcagaaagacatcagtactgaagagcagctaaggcgcctacaagaggagaagctttgcaaaatctgtatggatagaaatattgctatagtttttgttccttgtggacatctggtcacttgtaaacagtgtgcggaagcagttgacaaatgtcccatgtgctgcacagtcattacgttcaagcaaaaaatttttatgttttaa

SEQ ID NO: 10 - protein, 236 aa, Rattus norvegicus
MAEYDARIVTFGTWLYSVNKEQLARAGFYALGEGDKVKCFHCGGGLTDWKPSEDPWEQHAKWYPGCKYLLDEKGQEYINNIHLTHSLGESVVRTAEKTPSVTKKIDDTIFQNPMVQEAIRMGFNFKDIKKTMEEKLQTSGSNYLSLEVLIADLVSAQKDNSQDESSQTSLQKDISTEEQLRRLQEEKLCKICMDRNIAIVFVPCGHLVTCKQCAEAVDKCPMCCTVITFKQKIFMF

SEQ ID NO: 11 - DNA, 711 bp, Homo sapiens
  CDS (1)..(711)
atggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtcttaa

SEQ ID NO: 12 - protein, 236 aa, Homo sapiens
MADYEARIFTFGTWIYSVNKEQLARAGFYALGEGDKVKCFHCGGGLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 13 - DNA, 1443 bp, Mus musculus
  CDS (1)..(1443)
atggcccagagccctgtgtctgccgaggtcattcaccaggtggaagagtgtcttgatgaagacgagaaggagatgatgctcttcctgtgtagagatgtgactgagaacctggctgcacctaacgtcagggacctcctggatagcttaagtgagagaggccagctctcttttgctaccttggctgaattgctctacagagtgaggcggtttgaccttctcaagaggatcttgaagacagacaaagcaaccgtggaggaccacctgcgcagaaaccctcacctggtttctgattatagggtcctgctgatggagattggtgagagcttagatcagaacgatgtatcctccttagttttccttacaagggattacacaggcagaggcaagatagccaaggacaagagtttcttggatctggtgattgaattggagaaactgaatctaattgcttcagaccaattgaatttgttagaaaaatgcctgaagaacatccacagaatagacttgaacacaaagatccagaagtacacccagtccagccaaggagcaagatcaaatatgaatactctccaggcttcgctcccaaaattgagtatcaagtataactcaaggctccagaatgggcgaagtaaagagccaagatttgtggaataccgtgacagtcaaagaacactggtgaagacatccatccaggaatcaggagcttttttacctccgcacatccgtgaagagacttacaggatgcagagcaagcccctaggaatctgcttgatcattgattgtattggcaacgacacaaaatatcttcaagagaccttcacttccctgggctatcatatccagcttttcttgtttcccaagtcacatgacataacccagattgttcgccgatatgcaagtatggcccaacatcaagactatgacagctttgcatgtgttctggtgagcctaggaggctcccaaagcatgatgggcagagatcaagttcactcagggttctccttggatcatgtcaagaacatgttcacgggggacacgtgcccttctctcagagggaagccaaagctcttttttattcagaactatgagtcgttaggtagccagttggaagatagcagcctggaggtagatgggccatcaataaaaaatgtggactctaagcccctgcaacccagacactgcacaactcacccagaagctgatatcttttggagcctgtgcacagcagacgtatctcacttggagaagccctccagctcatcctctgtgtatctgcagaagctctcccagcagctgaagcaaggcaggagacgcccactcgtggacctccacgttgaactcatggacaaagtgtatgcgtggaacagtggtgtttcgtctaaggagaaatacagcctcagcctgcagcacactctgaggaagaaactcatcctggctcctacg

SEQ ID NO: 14 - protein, 481 aa, Mus musculus
MAQSPVSAEVIHQVEECLDEDEKEMMLFLCRDVTENLAAPNVRDLLDSLSERGQLSFATLAELLYRVRRFDLLKRILKTDKATVEDHLRRNPHLVSDYRVLLMEIGESLDQNDVSSLVFLTRDYTGRGKIAKDKSFLDLVIELEKLNLIASDQLNLLEKCLKNIHRIDLNTKIQKYTQSSQGARSNMNTLQASLPKLSIKYNSRLQNGRSKEPRFVEYRDSQRTLVKTSIQESGAFLPPHIREETYRMQSKPLGICLIIDCIGNDTKYLQETFTSLGYHIQLFLFPKSHDITQIVRRYASMAQHQDYDSFACVLVSLGGSQSMMGRDQVHSGFSLDHVKNMFTGDTCPSLRGKPKLFFIQNYESLGSQLEDSSLEVDGPSIKNVDSKPLQPRHCTTHPEADIFWSLCTADVSHLEKPSSSSSVYLQKLSQQLKQGRRRPLVDLHVELMDKVYAWNSGVSSKEKYSLSLQHTLRKKLILAPT

SEQ ID NO: 15 - DNA, 663 bp, Mus musculus
  CDS (1)..(663)
atggcccagagccctgtgtctgccgaggtcattcaccaggtggaagagtgtcttgatgaagacgagaaggagatgatgctcttcctgtgtagagatgtgactgagaacctggctgcacctaacgtcagggacctcctggatagcttaagtgagagaggccagctctcttttgctaccttggctgaattgctctacagagtgaggcggtttgaccttctcaagaggatcttgaagacagacaaagcaaccgtggaggaccacctgcgcagaaaccctcacctggtttctgattatagggtcctgctgatggagattggtgagagcttagatcagaacgatgtatcctccttagttttccttacaagggattacacaggcagaggcaagatagccaaggacaagagtttcttggatctggtgattgaattggagaaactgaatctaattgcttcagaccaattgaatttgttagaaaaatgcctgaagaacatccacagaatagacttgaacacaaagatccagaagtacacccagtccagccaaggagcaagatcaaatatgaatactctccaggcttcgctcccaaaattgagtatcaagtataactcaaggctccagaatgggcgaagtaaagagccaagatttgtggaataccgtgacagt

SEQ ID NO: 16 - protein, 221 aa, Mus musculus
MAQSPVSAEVIHQVEECLDEDEKEMMLFLCRDVTENLAAPNVRDLLDSLSERGQLSFATLAELLYRVRRFDLLKRILKTDKATVEDHLRRNPHLVSDYRVLLMEIGESLDQNDVSSLVFLTRDYTGRGKIAKDKSFLDLVIELEKLNLIASDQLNLLEKCLKNIHRIDLNTKIQKYTQSSQGARSNMNTLQASLPKLSIKYNSRLQNGRSKEPRFVEYRDS

SEQ ID NO: 17 - DNA, 930 bp, Mus musculus
  CDS (1)..(930)
atgatccagaagtacacccagtccagccaaggagcaagatcaaatatgaatactctccaggcttcgctcccaaaattgagtatcaagtataactcaaggctccagaatgggcgaagtaaagagccaagatttgtggaataccgtgacagtcaaagaacactggtgaagacatccatccaggaatcaggagcttttttacctccgcacatccgtgaagagacttacaggatgcagagcaagcccctaggaatctgcttgatcattgattgtattggcaacgacacaaaatatcttcaagagaccttcacttccctgggctatcatatccagcttttcttgtttcccaagtcacatgacataacccagattgttcgccgatatgcaagtatggcccaacatcaagactatgacagctttgcatgtgttctggtgagcctaggaggctcccaaagcatgatgggcagagatcaagttcactcagggttctccttggatcatgtcaagaacatgttcacgggggacacgtgcccttctctcagagggaagccaaagctcttttttattcagaactatgagtcgttaggtagccagttggaagatagcagcctggaggtagatgggccatcaataaaaaatgtggactctaagcccctgcaacccagacactgcacaactcacccagaagctgatatcttttggagcctgtgcacagcagacgtatctcacttggagaagccctccagctcatcctctgtgtatctgcagaagctctcccagcagctgaagcaaggcaggagacgcccactcgtggacctccacgttgaactcatggacaaagtgtatgcgtggaacagtggtgtttcgtctaaggagaaatacagcctcagcctgcagcacactctgaggaagaaactcatcctggctcctacg

SEQ ID NO: 18 - protein, 310 aa, Mus musculus
MIQKYTQSSQGARSNMNTLQASLPKLSIKYNSRLQNGRSKEPRFVEYRDSQRTLVKTSIQESGAFLPPHIREETYRMQSKPLGICLIIDCIGNDTKYLQETFTSLGYHIQLFLFPKSHDITQIVRRYASMAQHQDYDSFACVLVSLGGSQSMMGRDQVHSGFSLDHVKNMFTGDTCPSLRGKPKLFFIQNYESLGSQLEDSSLEVDGPSIKNVDSKPLQPRHCTTHPEADIFWSLCTADVSHLEKPSSSSSVYLQKLSQQLKQGRRRPLVDLHVELMDKVYAWNSGVSSKEKYSLSLQHTLRKKLILAPT

SEQ ID NO: 19 - DNA, 1821 bp, Rattus norvegicus
  CDS (223)..(255)
  CDS (331)..(1818)
atgcaggatctcgatcccgcgaaattaatacgactcactatagggagaccacaacggtttccctctagaaataattttgtttaactttaagaaggagatatacatatgcggggttctcatcatcatcatcatcatggtatggctagcatgactggtggacagcaaatgggtcgggatctgtacgacgatgacgataaggatcgatggggatccaagcttggctacggccgcaagaaagcgcgccagcgccgccgcggtggatccaccatgtccggctatccatatgacgtcccagactatgctggctccatggccggtaccggtctcgagatgacttttaacagttttgaaggatctagaactgttgtacctgcagacaccaataaggatgaagaatttgtagaagagtttaatagattaaaaacatttgctaacttcccaagcagcagtcctgtttcagcatcaacattggcgcgagcggggttcctctacactggtgaaggagacaccgtgcagtgtttcagttgtcacgcggcagtagatagatggcagtatggagactcagctgttggaagacacaggagaatatccccaaattgcagatttatcaatggtttttattttgaaaacggtgccacacagtctacatctcctggcatccaaaatggccagtacaaatctgaaaactgtgtgggaaacagaaatcattttgctcttgacaggccgtcggagactcatgcagattatctcctgagaactggacaggttgtagatatttcagataccatatacccgaggaacccggccatgtgtagtgaagaagccagaccgaagacgtttcagaactggccagactatgcccatttaagccccagagagttagctagtgctggactctactacacggggattgatgatcaagtgcaatgcttttgttgtggtggaaaactgaaaaattgggaaccctgtgaccgtgcctggtcagagcacaggagacactttcccaactgcttcttcgttttgggccggaatgttaatgttcgaagtgagtctggtgtgagttcagataggaatttcccaaattcaacaaattctccaagaaatccagccatggcagaatatgacgcacggatcgttacttttggaacatggctatactcagttaacaaggagcagcttgcaagagctggattttatgctttaggtgaaggtgataaagtgaagtgctttcactgtggaggagggctcacggattggaagccaagtgaagacccttgggaacagcatgctaagtggtatccagggtgtaaatatctattggatgagaagggacaagaatatataaataatattcatttaacccattcacttggggaatctgtggtaagaactgctgaaaaaacaccatcagtaactaaaaaaatcgatgataccatcttccagaatcctatggtgcaagaagctatacgaatgggattcaacttcaaggacatcaagaaaacaatggaagaaaagctccaaacatctgggagcaactatctatcacttgaggttctgattgcagatcttgtgagtgctcagaaagataattcgcaggatgagtcaagtcagacttcattgcagaaagacatcagtactgaagagcagctaaggcgcctacaagaggagaagctttgcaaaatctgtatggatagaaatattgctatagtttttgttccttgtggacatctggtcacttgtaaacagtgtgcggaagcagttgacaaatgtcccatgtgctgcacagtcattacgttcaagcaaaaaatttttatgttttaa

SEQ ID NO: 20 - protein, 507 aa, Rattus norvegicus
YGRKKARQRRRMTFNSFEGSRTVVPADTNKDEEFVEEFNRLKTFANFPSSSPVSASTLARAGFLYTGEGDTVQCFSCHAAVDRWQYGDSAVGRHRRISPNCRFINGFYFENGATQSTSPGIQNGQYKSENCVGNRNHFALDRPSETHADYLLRTGQVVDISDTIYPRNPAMCSEEARPKTFQNWPDYAHLSPRELASAGLYYTGIDDQVQCFCCGGKLKNWEPCDRAWSEHRRHFPNCFFVLGRNVNVRSESGVSSDRNFPNSTNSPRNPAMAEYDARIVTFGTWLYSVNKEQLARAGFYALGEGDKVKCFHCGGGLTDWKPSEDPWEQHAKWYPGCKYLLDEKGQEYINNIHLTHSLGESVVRTAEKTPSVTKKIDDTIFQNPMVQEAIRMGFNFKDIKKTMEEKLQTSGSNYLSLEVLIADLVSAQKDNSQDESSQTSLQKDISTEEQLRRLQEEKLCKICMDRNIAIVFVPCGHLVTCKQCAEAVDKCPMCCTVITFKQKIFMF

SEQ ID NO: 21 - DNA, 1824 bp, Homo sapiens
  CDS (223)..(255)
  CDS (331)..(1821)
atgcaggatctcgatcccgcgaaattaatacgactcactatagggagaccacaacggtttccctctagaaataattttgtttaactttaagaaggagatatacatatgcggggttctcatcatcatcatcatcatggtatggctagcatgactggtggacagcaaatgggtcgggatctgtacgacgatgacgataaggatcgatggggatccaagcttggctacggccgcaagaaagcgcgccagcgccgccgcggtggatccaccatgtccggctatccatatgacgtcccagactatgctggctccatggccggtaccggtctcgagatgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagatagatggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtcttaa

SEQ ID NO: 22 - protein, 508 aa, Homo sapiens
YGRKKARQRRRMTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 23 - DNA, 741 bp, Rattus norvegicus
  CDS (223)..(255)
  CDS (331)..(741)
atgcaggatctcgatcccgcgaaattaatacgactcactatagggagaccacaacggtttccctctagaaataattttgtttaactttaagaaggagatatacatatgcggggttctcatcatcatcatcatcatggtatggctagcatgactggtggacagcaaatgggtcgggatctgtacgacgatgacgataaggatcgatggggatccaagcttggctacggccgcaagaaagcgcgccagcgccgccgcggtggatccaccatgtccggctatccatatgacgtcccagactatgctggctccatggccggtaccggtctcgagagaaatcattttgctcttgacaggccgtcggagactcatgcagattatctcctgagaactggacaggttgtagatatttcagataccatatacccgaggaacccggccatgtgtagtgaagaagccagaccgaagacgtttcagaactggccagactatgcccatttaagccccagagagttagctagtgctggactctactacacggggattgatgatcaagtgcaatgcttttgttgtggtggaaaactgaaaaattgggaaccctgtgaccgtgcctggtcagagcacaggagacactttcccaactgcttcttcgttttgggccggaatgttaatgttcgaagtgagtctggtgtgagttcagataggaatttcccaaattcaacaaattctccaagaaatccagcc

SEQ ID NO: 24 - protein, 148 aa, Rattus norvegicus
YGRKKARQRRRRNHFALDRPSETHADYLLRTGQVVDISDTIYPRNPAMCSEEARPKTFQNWPDYAHLSPRELASAGLYYTGIDDQVQCFCCGGKLKNWEPCDRAWSEHRRHFPNCFFVLGRNVNVRSESGVSSDRNFPNSTNSPRNPA

SEQ ID NO: 25 - DNA, 1041 bp, Rattus norvegicus
  CDS (223)..(255)
  CDS (331)..(1038)
atgcaggatctcgatcccgcgaaattaatacgactcactatagggagaccacaacggtttccctctagaaataattttgtttaactttaagaaggagatatacatatgcggggttctcatcatcatcatcatcatggtatggctagcatgactggtggacagcaaatgggtcgggatctgtacgacgatgacgataaggatcgatggggatccaagcttggctacggccgcaagaaagcgcgccagcgccgccgcggtggatccaccatgtccggctatccatatgacgtcccagactatgctggctccatggccggtaccggtctcgagatggcagaatatgacgcacggatcgttacttttggaacatggctatactcagttaacaaggagcagcttgcaagagctggattttatgctttaggtgaaggtgataaagtgaagtgctttcactgtggaggagggctcacggattggaagccaagtgaagacccttgggaacagcatgctaagtggtatccagggtgtaaatatctattggatgagaagggacaagaatatataaataatattcatttaacccattcacttggggaatctgtggtaagaactgctgaaaaaacaccatcagtaactaaaaaaatcgatgataccatcttccagaatcctatggtgcaagaagctatacgaatgggattcaacttcaaggacatcaagaaaacaatggaagaaaagctccaaacatctgggagcaactatctatcacttgaggttctgattgcagatcttgtgagtgctcagaaagataattcgcaggatgagtcaagtcagacttcattgcagaaagacatcagtactgaagagcagctaaggcgcctacaagaggagaagctttgcaaaatctgtatggatagaaatattgctatagtttttgttccttgtggacatctggtcacttgtaaacagtgtgcggaagcagttgacaaatgtcccatgtgctgcacagtcattacgttcaagcaaaaaatttttatgttttaa

SEQ ID NO: 26 - protein, 247 aa, Rattus norvegicus
YGRKKARQRRRMAEYDARIVTFGTWLYSVNKEQLARAGFYALGEGDKVKCFHCGGGLTDWKPSEDPWEQHAKWYPGCKYLLDEKGQEYINNIHLTHSLGESVVRTAEKTPSVTKKIDDTIFQNPMVQEAIRMGFNFKDIKKTMEEKLQTSGSNYLSLEVLIADLVSAQKDNSQDESSQTSLQKDISTEEQLRRLQEEKLCKICMDRNIAIVFVPCGHLVTCKQCAEAVDKCPMCCTVITFKQKIFMF

SEQ ID NO: 27 - DNA, 1041 bp, Homo sapiens
  CDS (223)..(255)
  CDS (331)..(1038)
atgcaggatctcgatcccgcgaaattaatacgactcactatagggagaccacaacggtttccctctagaaataattttgtttaactttaagaaggagatatacatatgcggggttctcatcatcatcatcatcatggtatggctagcatgactggtggacagcaaatgggtcgggatctgtacgacgatgacgataaggatcgatggggatccaagcttggctacggccgcaagaaagcgcgccagcgccgccgcggtggatccaccatgtccggctatccatatgacgtcccagactatgctggctccatggccggtaccggtctcgagatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtcttaa

SEQ ID NO: 28 - protein, 247 aa, Homo sapiens
YGRKKARQRRRMADYEARIFTFGTWIYSVNKEQLARAGFYALGEGDKVKCFHCGGGLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 29 - DNA, 1773 bp, Mus musculus
  CDS (223)..(255)
  CDS (331)..(1773)
atgcaggatctcgatcccgcgaaattaatacgactcactatagggagaccacaacggtttccctctagaaataattttgtttaactttaagaaggagatatacatatgcggggttctcatcatcatcatcatcatggtatggctagcatgactggtggacagcaaatgggtcgggatctgtacgacgatgacgataaggatcgatggggatccaagcttggctacggccgcaagaaagcgcgccagcgccgccgcggtggatccaccatgtccggctatccatatgacgtcccagactatgctggctccatggccggtaccggtctcgagatggcccagagccctgtgtctgccgaggtcattcaccaggtggaagagtgtcttgatgaagacgagaaggagatgatgctcttcctgtgtagagatgtgactgagaacctggctgcacctaacgtcagggacctcctggatagcttaagtgagagaggccagctctcttttgctaccttggctgaattgctctacagagtgaggcggtttgaccttctcaagaggatcttgaagacagacaaagcaaccgtggaggaccacctgcgcagaaaccctcacctggtttctgattatagggtcctgctgatggagattggtgagagcttagatcagaacgatgtatcctccttagttttccttacaagggattacacaggcagaggcaagatagccaaggacaagagtttcttggatctggtgattgaattggagaaactgaatctaattgcttcagaccaattgaatttgttagaaaaatgcctgaagaacatccacagaatagacttgaacacaaagatccagaagtacacccagtccagccaaggagcaagatcaaatatgaatactctccaggcttcgctcccaaaattgagtatcaagtataactcaaggctccagaatgggcgaagtaaagagccaagatttgtggaataccgtgacagtcaaagaacactggtgaagacatccatccaggaatcaggagcttttttacctccgcacatccgtgaagagacttacaggatgcagagcaagcccctaggaatctgcttgatcattgattgtattggcaacgacacaaaatatcttcaagagaccttcacttccctgggctatcatatccagcttttcttgtttcccaagtcacatgacataacccagattgttcgccgatatgcaagtatggcccaacatcaagactatgacagctttgcatgtgttctggtgagcctaggaggctcccaaagcatgatgggcagagatcaagttcactcagggttctccttggatcatgtcaagaacatgttcacgggggacacgtgcccttctctcagagggaagccaaagctcttttttattcagaactatgagtcgttaggtagccagttggaagatagcagcctggaggtagatgggccatcaataaaaaatgtggactctaagcccctgcaacccagacactgcacaactcacccagaagctgatatcttttggagcctgtgcacagcagacgtatctcacttggagaagccctccagctcatcctctgtgtatctgcagaagctctcccagcagctgaagcaaggcaggagacgcccactcgtggacctccacgttgaactcatggacaaagtgtatgcgtggaacagtggtgtttcgtctaaggagaaatacagcctcagcctgcagcacactctgaggaagaaactcatcctggctcctacg

SEQ ID NO: 30 - protein, 492 aa, Mus musculus
YGRKKARQRRRMAQSPVSAEVIHQVEECLDEDEKEMMLFLCRDVTENLAAPNVRDLLDSLSERGQLSFATLAELLYRVRRFDLLKRILKTDKATVEDHLRRNPHLVSDYRVLLMEIGESLDQNDVSSLVFLTRDYTGRGKIAKDKSFLDLVIELEKLNLIASDQLNLLEKCLKNIHRIDLNTKIQKYTQSSQGARSNMNTLQASLPKLSIKYNSRLQNGRSKEPRFVEYRDSQRTLVKTSIQESGAFLPPHIREETYRMQSKPLGICLIIDCIGNDTKYLQETFTSLGYHIQLFLFPKSHDITQIVRRYASMAQHQDYDSFACVLVSLGGSQSMMGRDQVHSGFSLDHVKNMFTGDTCPSLRGKPKLFFIQNYESLGSQLEDSSLEVDGPSIKNVDSKPLQPRHCTTHPEADIFWSLCTADVSHLEKPSSSSSVYLQKLSQQLKQGRRRPLVDLHVELMDKVYAWNSGVSSKEKYSLSLQHTLRKKLILAPT

SEQ ID NO: 31 - DNA, 993 bp, Mus musculus
  CDS (223)..(255)
  CDS (331)..(993)
atgcaggatctcgatcccgcgaaattaatacgactcactatagggagaccacaacggtttccctctagaaataattttgtttaactttaagaaggagatatacatatgcggggttctcatcatcatcatcatcatggtatggctagcatgactggtggacagcaaatgggtcgggatctgtacgacgatgacgataaggatcgatggggatccaagcttggctacggccgcaagaaagcgcgccagcgccgccgcggtggatccaccatgtccggctatccatatgacgtcccagactatgctggctccatggccggtaccggtctcgagatggcccagagccctgtgtctgccgaggtcattcaccaggtggaagagtgtcttgatgaagacgagaaggagatgatgctcttcctgtgtagagatgtgactgagaacctggctgcacctaacgtcagggacctcctggatagcttaagtgagagaggccagctctcttttgctaccttggctgaattgctctacagagtgaggcggtttgaccttctcaagaggatcttgaagacagacaaagcaaccgtggaggaccacctgcgcagaaaccctcacctggtttctgattatagggtcctgctgatggagattggtgagagcttagatcagaacgatgtatcctccttagttttccttacaagggattacacaggcagaggcaagatagccaaggacaagagtttcttggatctggtgattgaattggagaaactgaatctaattgcttcagaccaattgaatttgttagaaaaatgcctgaagaacatccacagaatagacttgaacacaaagatccagaagtacacccagtccagccaaggagcaagatcaaatatgaatactctccaggcttcgctcccaaaattgagtatcaagtataactcaaggctccagaatgggcgaagtaaagagccaagatttgtggaataccgtgacagt

SEQ ID NO: 32 - protein, 232 aa, Mus musculus
YGRKKARQRRRMAQSPVSAEVIHQVEECLDEDEKEMMLFLCRDVTENLAAPNVRDLLDSLSERGQLSFATLAELLYRVRRFDLLKRILKTDKATVEDHLRRNPHLVSDYRVLLMEIGESLDQNDVSSLVFLTRDYTGRGKIAKDKSFLDLVIELEKLNLIASDQLNLLEKCLKNIHRIDLNTKIQKYTQSSQGARSNMNTLQASLPKLSIKYNSRLQNGRSKEPRFVEYRDS

SEQ ID NO: 33 - DNA, 1260 bp, Mus musculus
  CDS (223)..(255)
  CDS (331)..(1260)
atgcaggatctcgatcccgcgaaattaatacgactcactatagggagaccacaacggtttccctctagaaataattttgtttaactttaagaaggagatatacatatgcggggttctcatcatcatcatcatcatggtatggctagcatgactggtggacagcaaatgggtcgggatctgtacgacgatgacgataaggatcgatggggatccaagcttggctacggccgcaagaaagcgcgccagcgccgccgcggtggatccaccatgtccggctatccatatgacgtcccagactatgctggctccatggccggtaccggtctcgagatgatccagaagtacacccagtccagccaaggagcaagatcaaatatgaatactctccaggcttcgctcccaaaattgagtatcaagtataactcaaggctccagaatgggcgaagtaaagagccaagatttgtggaataccgtgacagtcaaagaacactggtgaagacatccatccaggaatcaggagcttttttacctccgcacatccgtgaagagacttacaggatgcagagcaagcccctaggaatctgcttgatcattgattgtattggcaacgacacaaaatatcttcaagagaccttcacttccctgggctatcatatccagcttttcttgtttcccaagtcacatgacataacccagattgttcgccgatatgcaagtatggcccaacatcaagactatgacagctttgcatgtgttctggtgagcctaggaggctcccaaagcatgatgggcagagatcaagttcactcagggttctccttggatcatgtcaagaacatgttcacgggggacacgtgcccttctctcagagggaagccaaagctcttttttattcagaactatgagtcgttaggtagccagttggaagatagcagcctggaggtagatgggccatcaataaaaaatgtggactctaagcccctgcaacccagacactgcacaactcacccagaagctgatatcttttggagcctgtgcacagcagacgtatctcacttggagaagccctccagctcatcctctgtgtatctgcagaagctctcccagcagctgaagcaaggcaggagacgcccactcgtggacctccacgttgaactcatggacaaagtgtatgcgtggaacagtggtgtttcgtctaaggagaaatacagcctcagcctgcagcacactctgaggaagaaactcatcctggctcctacg

SEQ ID NO: 34 - protein, 321 aa, Mus musculus
YGRKKARQRRRMIQKYTQSSQGARSNMNTLQASLPKLSIKYNSRLQNGRSKEPRFVEYRDSQRTLVKTSIQESGAFLPPHIREETYRMQSKPLGICLIIDCIGNDTKYLQETFTSLGYHIQLFLFPKSHDITQIVRRYASMAQHQDYDSFACVLVSLGGSQSMMGRDQVHSGFSLDHVKNMFTGDTCPSLRGKPKLFFIQNYESLGSQLEDSSLEVDGPSIKNVDSKPLQPRHCTTHPEADIFWSLCTADVSHLEKPSSSSSVYLQKLSQQLKQGRRRPLVDLHVELMDKVYAWNSGVSSKEKYSLSLQHTLRKKLILAPT

SEQ ID NO: 35 - DNA, 360 bp, Rattus norvegicus
  CDS (1)..(360)
ctattggatgagaagggacaagaatatataaataatattcatttaacccattcacttggggaatctgtggtaagaactgctgaaaaaacaccatcagtaactaaaaaaatcgatgataccatcttccagaatcctatggtgcaagaagctatacgaatgggattcaacttcaaggacatcaagaaaacaatggaagaaaagctccaaacatctgggagcaactatctatcacttgaggttctgattgcagatcttgtgagtgctcagaaagataattcgcaggatgagtcaagtcagacttcattgcagaaagacatcagtactgaagagcagctaaggcgcctacaagaggagaagctt

SEQ ID NO: 36 - protein, 120 aa, Rattus norvegicus
LLDEKGQEYINNIHLTHSLGESVVRTAEKTPSVTKKIDDTIFQNPMVQEAIRMGFNFKDIKKTMEEKLQTSGSNYLSLEVLIADLVSAQKDNSQDESSQTSLQKDISTEEQLRRLQEEKL

SEQ ID NO: 37 - DNA, 360 bp, Homo sapiens
  CDS (1)..(360)
ctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctt

SEQ ID NO: 38 - protein, 120 aa, Homo sapiens
LLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKL

SEQ ID NO: 39 - DNA, 690 bp, Rattus norvegicus
  CDS (223)..(255)
  CDS (331)..(690)
atgcaggatctcgatcccgcgaaattaatacgactcactatagggagaccacaacggtttccctctagaaataattttgtttaactttaagaaggagatatacatatgcggggttctcatcatcatcatcatcatggtatggctagcatgactggtggacagcaaatgggtcgggatctgtacgacgatgacgataaggatcgatggggatccaagcttggctacggccgcaagaaagcgcgccagcgccgccgcggtggatccaccatgtccggctatccatatgacgtcccagactatgctggctccatggccggtaccggtctcgagctattggatgagaagggacaagaatatataaataatattcatttaacccattcacttggggaatctgtggtaagaactgctgaaaaaacaccatcagtaactaaaaaaatcgatgataccatcttccagaatcctatggtgcaagaagctatacgaatgggattcaacttcaaggacatcaagaaaacaatggaagaaaagctccaaacatctgggagcaactatctatcacttgaggttctgattgcagatcttgtgagtgctcagaaagataattcgcaggatgagtcaagtcagacttcattgcagaaagacatcagtactgaagagcagctaaggcgcctacaagaggagaagctt

SEQ ID NO: 40 - protein, 131 aa, Rattus norvegicus
YGRKKARQRRRLLDEKGQEYINNIHLTHSLGESVVRTAEKTPSVTKKIDDTIFQNPMVQEAIRMGFNFKDIKKTMEEKLQTSGSNYLSLEVLIADLVSAQKDNSQDESSQTSLQKDISTEEQLRRLQEEKL

SEQ ID NO: 41 - DNA, 690 bp, Homo sapiens
  CDS (223)..(255)
  CDS (331)..(690)
atgcaggatctcgatcccgcgaaattaatacgactcactatagggagaccacaacggtttccctctagaaataattttgtttaactttaagaaggagatatacatatgcggggttctcatcatcatcatcatcatggtatggctagcatgactggtggacagcaaatgggtcgggatctgtacgacgatgacgataaggatcgatggggatccaagcttggctacggccgcaagaaagcgcgccagcgccgccgcggtggatccaccatgtccggctatccatatgacgtcccagactatgctggctccatggccggtaccggtctcgagctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctt

SEQ ID NO: 42 - protein, 131 aa, Homo sapiens
YGRKKARQRRRLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKL

SEQ ID NO: 43 - DNA, 414 bp, Homo sapiens
  CDS (1)..(414)
agagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatcc

SEQ ID NO: 44 - protein, 138 aa, Homo sapiens
RDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPS

SEQ ID NO: 45 - DNA, 744 bp, Homo sapiens
  CDS (223)..(255)
  CDS (331)..(744)
atgcaggatctcgatcccgcgaaattaatacgactcactatagggagaccacaacggtttccctctagaaataattttgtttaactttaagaaggagatatacatatgcggggttctcatcatcatcatcatcatggtatggctagcatgactggtggacagcaaatgggtcgggatctgtacgacgatgacgataaggatcgatggggatccaagcttggctacggccgcaagaaagcgcgccagcgccgccgcggtggatccaccatgtccggctatccatatgacgtcccagactatgctggctccatggccggtaccggtctcgagagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatcc

SEQ ID NO: 46 - protein, 149 aa, Homo sapiens
YGRKKARQRRRRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPS

SEQ ID NO: 47 - DNA, 32 bp, Artificial Sequence
  Description of Artificial Sequence Synthetic oligonucleotide
gggctcgagatgacttttaacagttttgaagg

SEQ ID NO: 48 - DNA, 35 bp, Artificial Sequence
  Description of Artificial Sequence Synthetic oligonucleotide
ggggaattcttaaaacataaaaatttttttgcttg

SEQ ID NO: 49 - protein, 11 aa, Artificial Sequence
  Description of Artificial Sequence Synthetic PTD tag
YGRKKRRQRRR

SEQ ID NO: 50 - protein, 9 aa, Artificial Sequence
  Description of Artificial Sequence Synthetic hemagglutinin (HA) tag
YPYDVPDVA

SEQ ID NO: 51 - DNA, 24 bp, Artificial Sequence
  Description of Artificial Sequence Synthetic oligonucleotide
agaaatcattttgctcttgacagg

SEQ ID NO: 52 - DNA, 25 bp, Artificial Sequence
  Description of Artificial Sequence Synthetic oligonucleotide
caagcaaaaaatttttatgttttaa

SEQ ID NO: 53 - DNA, 21 bp, Artificial Sequence
  Description of Artificial Sequence Synthetic oligonucleotide
atggcagaatatgacgcacgg

SEQ ID NO: 54 - DNA, 25 bp, Artificial Sequence
  Description of Artificial Sequence Synthetic oligonucleotide
caagcaaaaaatttttatgttttaa

SEQ ID NO: 55 - DNA, 21 bp, Artificial Sequence
  Description of Artificial Sequence Synthetic oligonucleotide
atggcagattatgaagcacgg

SEQ ID NO: 56 - DNA, 25 bp, Artificial Sequence
  Description of Artificial Sequence Synthetic oligonucleotide
caagcaaaaaatttttatgtcttaa

SEQ ID NO: 57 - DNA, 22 bp, Artificial Sequence
  Description of Artificial Sequence Synthetic consensus binding sequences of NF-kB
agttgaggggactttcccaggc

SEQ ID NO: 58 - DNA, 21 bp, Artificial Sequence
  Description of Artificial Sequence Synthetic consensus binding sequences of AP1
cgcttgatgagtcagccggaa

SEQ ID NO: 59 - DNA, 22 bp, Artificial Sequence
  Description of Artificial Sequence Synthetic consensus binding sequences of OCT-1
tgtcgaatgcaaatcactagaa

SEQ ID NO: 60 - DNA, 25 bp, Artificial Sequence
  Description of Artificial Sequence Synthetic consensus binding sequences of TFIID
gcagagcatataaggtgaggtagga